(19) United States
(12) Patent Application Publication
(10) Pub. No.: US 2026/0357349 A1
(43) Pub. Date: Nov. 12, 2026

(54) METHODS OF IDENTIFYING AND TREATING CANCER

(71) Applicants: Agency for Science, Technology and Research, Singapore (SG); National University of Singapore, Singapore (SG); Singapore Health Services Pte Ltd, Singapore (SG)

(72) Inventors: Xiaosai YAO, Singapore (SG); Jing Han HONG, Singapore (SG); Amrita Mandar MANGALVEDHEKAR, Singapore (SG); Shu Wen Michelle NG, Singapore (SG); Bin Tean TEH, Singapore (SG)

(73) Assignees: Agency for Science, Technology and Research, Singapore (SG); National University of Singapore, Singapore (SG); Singapore Health Services Pte Ltd, Singapore (SG)

(21) Appl. No.: 19/112,571

(22) PCT Filed: Jun. 26, 2023
(86) PCT No.: PCT/SG2023/050450

§ 371 Date: Mar. 17, 2025

(30) Foreign Application Priority Data
Sep. 26, 2022 (SG) 10202251154K

(51) Int. Cl. C12Q 1/6886; A61K 31/5377; A61K 31/69; A61P 35/00; G01N 33/575; G01N 33/5754

(52) U.S. Cl. CPC C12Q 1/6886; A61K 31/5377; A61K 31/69; A61P 35/00; G01N 33/5754; G01N 33/57585; C12Q 2600/106; G01N 2333/4704

(57) ABSTRACT

The present invention relates, in general terms, to cancer biomarkers. Disclosed herein are methods of identifying cancers that are suitable for NF-κB inhibition therapy, and methods of selecting subjects for the same, comprising detecting an indicator that is indicative of a level or activity of Protein polybromo-1 (PB1) in a sample obtained from the subject, wherein the cancer is identified as being likely to be responsive to the inhibitor of NF-κB signalling when the level or activity of PB1 indicated by the indicator corresponds to an absence or decreased level or activity of PB1 in the sample as compared to a control.

### TECHNICAL FIELD

The present invention relates, in general terms, to cancer biomarkers. Disclosed herein are methods of identifying cancers that are suitable for NF-κB inhibition therapy, and methods of selecting subjects for the same, using a cancer biomarker.

### BACKGROUND

Polybromo-1 (PB1) is an accessory subunit of the polybromo BRG1-associated factor (PBAF) subclass of the SWI/SNF (SWItch/Sucrose Non-Fermentable) nucleosome remodeling complex, which regulates chromatin accessibility by sliding, depositing or evicting nucleosomes. PB1 is the second most frequently mutated gene in kidney tumorigenesis after Von Hippel-Lindau (VHL), occurring at a frequency of 30-40% in clear cell renal cell carcinomas (ccRCC).

Even though biochemical studies have established the interactions of PB1 with nucleosomes, the role of PB1 in ccRCC tumorigenesis has not been examined previously. As a tumor suppressor, PB1 has been shown to regulate divergent processes including the cell cycle, DNA damage repair, interferon response and stress response. For example, PB1 can negatively regulate cell cycle progression, silence transcription around DNA double stranded breaks (DSBs), and promote cohesion formation at centromeres and thus prevent genome instability and aneuploidy.

Several studies have highlighted the pro-tumorigenic role of PB1 loss, but pharmaceutical interventions which target PB1 loss remain obscure. PB1-deficient tumors are sensitive to anti-angiogenic therapies. PB1 loss is correlated with improved checkpoint therapy response in renal cell carcinoma (RCC) patients only in the specific setting of post-VEGF tyrosine kinase inhibitor immune checkpoint blockade (TKI ICB) monotherapy, with a medium effect size association that is likely to be insufficient for a clinical biomarker.

Further mechanistic insight into tumorigenic pathways consequent to PB1 loss, especially in cancers also characterised by VHL deficiency, is necessary to guide current RCC treatment and the development of new therapeutic targets or diagnostic or prognostic biomarkers for treatment.

It would be desirable to overcome or ameliorate at least one of the above-described problems, or at least to provide a useful alternative.

### SUMMARY

Disclosed herein is a method of identifying a cancer that is likely to be responsive to treatment with an inhibitor of NF-κB signalling in a subject, the method comprising detecting an indicator that is indicative of a level or activity of Protein polybromo-1 (PB1) in a sample obtained from the subject, wherein the cancer is identified as being likely to be responsive to the inhibitor of NF-κB signalling when the level or activity of PB1 indicated by the indicator corresponds to an absence or decreased level or activity of PB1 in the sample as compared to a control.

Also disclosed herein is a method of treating a cancer in a subject, the method comprising a) detecting an indicator that is indicative of a level or activity of PB1 in a sample obtained from the subject, wherein the cancer is identified as being likely to be responsive to the inhibitor of NF-κB signalling when the level or activity of PB1 indicated by the indicator corresponds to an absence or decreased level or activity of PB1 in the sample as compared to a control; and b) administering an inhibitor of NF-κB signalling to the subject when the subject is found likely to be responsive to treatment with the inhibitor of NF-κB signalling.

Disclosed herein is a method of inhibiting proliferation of a cancer cell, the method comprising contacting the cancer cell with an inhibitor of NF-κB signalling, wherein PB1 is absent in the cancer cell or wherein the cancer cell has a decreased level or activity of PB1 as compared to a control.

Disclosed herein is a method of selecting a cancer subject for treatment with an inhibitor of NF-κB signalling, the method comprising detecting an indicator that is indicative of a level or activity of PB1 in a sample obtained from the subject, wherein the cancer is identified as being likely to be responsive to the inhibitor of NF-κB signalling when the level or activity of PB1 indicated by the indicator corresponds to an absence or decreased level or activity of PB1 in the sample as compared to a control; and b) selecting the cancer subject found likely to be responsive to treatment with the inhibitor of NF-κB signalling for treatment with the inhibitor of NF-κB signalling.

Disclosed herein is a method of stratifying a cancer subject as a likely responder or non-responder to an inhibitor of NF-κB signalling, the method comprising detecting an indicator that is indicative of a level or activity of PB1 in a sample obtained from the subject, wherein the cancer is identified as being likely to be responsive to the inhibitor of NF-κB signalling when the level or activity of PB1 indicated by the indicator corresponds to an absence or decreased level or activity of PB1 in the sample as compared to a control, thereby stratifying the cancer subject as a likely responder or non-responder to the inhibitor of NF-κB signalling.

### BRIEF DESCRIPTION OF DRAWINGS

Embodiments of the present invention are hereafter described, by way of non-limiting example only, with reference to the accompanying drawings in which:

FIG. 1 shows that PBRM1 loss is pro-tumorigenic. a) VHL and PBRM1 mutation status in a panel of normal kidney epithelial cells and ccRCC cell lines used in this study. PCS400 and HK2-normal kidney epithelial cell lines, 786-0 and 769-P-VHL-deficient/PBRM1-proficient ccRCC lines and NCC432 and NCC2112-VHL/PBRM1 double deficient ccRCC lines. b) Immunoblots of VHL and PBRM1 (results representative of 2 biological replicates. c) Protein levels of PBRM1 in 5 cell lines, in which we either knocked out PBRM1 or restored its expression. Relative PBRM1 protein levels normalized to beta-actin are indicated. d) In vitro colony formation (measured as the percentage of total plate area) of 4 isogenic cell lines under 10% or low serum (n=3 biological replicates). Data are presented as mean values+/−SD. * p-value <0.05 (two-sided t-test). e) In vivo tumor formation of PBRM1 wildtype (WT) or knockout (KO) 786-O cells, and PBRM1-deficient NCC432 (EV-empty vector) and PBRM1-restored (PBRM1) isogenic pair (n=6 mice per group). Tumor volume plots represent mean values+/−SEM and tumor weight plots represent mean values+/−SD (two-sided t-test).

FIG. 2 shows that PBRM1-deficient PBAF complexes disengage BRD7 but retain ARID2 and SMARCA4. a) Co-immunoprecipitation of PBRM1 in isogenic pairs of 786-O cells with and without PBRM1 (results representative of 2 biological replicates). b) ChIP-mass spectrometry of PBRM1 in isogenic pairs of 786-O cells (n=2 biological replicates). c) Co-immunoprecipitation of SMARCA4 in isogenic pairs of 786-O cells (results representative of 2 biological replicates). d) ChIP-mass spectrometry of SMARCA4 in isogenic pairs of 786-O cells. Total intensity merged from 3 technical replicates. e) Co-immunoprecipitation of BRD7 and ARID2 in isogenic pairs of 786-O cells (results representative of 2 biological replicates).

FIG. 3 shows that PBRM1 binds to promoter-proxy regions. a) PBRM1 ChIP-seq of PBRM1 wildtype (WT) or knockout (KO) 786-O cells, and in PBRM1-deficient NCC2112 (EV-empty vector) and PBRM1-restored (PBRM1) isogenic pair. Distribution of the binding regions of (b) PBRM1 and (c) ARID1A in 786-O cells. d) Nucleosome positioning determined from ATAC-seq of 786-O cells is superimposed on the ChIP-seq of PBRM1. e) ChIP-seq profiles of selected components of the SWI/SNF complex and RNA polymerase II aligned at transcription start sites (TSS). f) Close-up view of the ChIP-seq profiles of PBAF components at +1 Kb of TSS. g) Unsupervised hierarchical clustering of ChIP-seq profiles of the SWI/SNF components. h, i) ChIP-seq profiles of histone marks at +3 Kb of TSS. j) Unsupervised hierarchical clustering of ChIP-seq profiles of histone marks with PBRM1 ChIP-seq.

FIG. 4 shows that PBRM1-deficient PBAF complexes are mistargeted. a) Differential ChIP-seq peaks of the various SWI/SNF subunits between parental and PBRM1-deficient 786-O cells were identified by Diffbind (n=2 biological replicates). Significantly altered regions are colored in pink (q<0.01). Numbers of significantly differential peaks (up and down) are shown. A positive log fold change refers to gains in PBRM1 KO over WT. b) ChIP-seq profiles of SWI/SNF complex components, histone marks and Pol II in PBRM1 WT vs. KO 786-O cells at gained SMARCA4 sites (black-WT; red-KO). c) Genomic distribution of differential and unchanged SMARCA4 peaks. Promoter regions were defined as H3K27ac+/H3K4me3+/TSS+2.5 kb whereas enhancers were defined as H3K27ac+/H3K4me1+/promoter-. d) Overlaps between gained SMARCA4 regions and lost PBRM1, gained ARID2 or gained BRD7 regions were calculated using ChIPpeakAnno (hypergeometric tests).

FIG. 5 shows that PBRM1 loss activates NF-κB pathway. a) Shown is the intersection between upregulated genes after PBRM1 KO and downregulated genes after PBRM1 re-expression in PBRM1 KO to define truly derepressed targets of PBRM1 in 786-O cells. The same was performed for downregulated targets of PBRM1 KO. b) Hallmark gene set enrichment of de-repressed targets of PBRM1 defined in a. Hypergeometric test and p-value corrected for multiple comparisons. c) Gene expression changes of NF-κB targets after PBRM1 KO in 786-O cells measured by RNA-seq (n=2 biological replicates). d) Gene expression changes of selected NF-κB targets in PBRM1-deficient NCC432 cells expressing empty vector control or PBRM1 measured by RNA-seq (n=2 biological replicates). e) Gene expression changes of NF-κB targets in genetic mouse model of single wildtype (WT), double knockouts of VHL and PBRM1 (DKO) or single knockouts of PBRM1 or VHL measured by microarray (n=4 biological replicates). f) Normalized gene expression of selected upregulated NF-κB targets in 786-O PBRM1 WT and KO cells after TNFα stimulation (0, 1 and 10 ng/ml) for 3 hours measured by RT-qPCR (n=3 technical replicates representative of 2 biological replicates). * p-value<0.05 (two-sided t-test). Data are presented as mean values+/−SD. g) Gene expression changes of NF-κB target genes after shRNA-mediated knockdown of NF-κB subunits measured by RT-qPCR (n=3 technical replicates representative of 2 biological replicates). * p-value<0.05 (two-sided t-test). Data are presented as mean values+/−SD. h) RELA targets are defined by concomitant RELA occupancy and downregulated gene expression after shRELA (p<0.05 and log 2FC<−0.5). The RELA targets are further overlapped with upregulated and downregulated genes after PBRM1 KO (p<0.05 and |log 2FC|>0.5). P-value of the overlap was computed by hypergeometric test.

FIG. 6 shows that NF-κB activation requires SMARCA4 and ARID2. a) 786-0 parental and PBRM1 KO cells were treated with SMARCA2/4 ATPase inhibitor at 10, 20 and 50 μM for 3 days and the expression of NF-κB targets was evaluated by RT-qPCR (n=3 technical replicates representative of 2 biological replicates). Data are presented as mean values+/−SD. b, c) 786-O parental and PBRM1 KO cells were treated with 10 μM of SMARCA2/4 ATPase inhibitor for 24 hours and transcriptomic changes were evaluated by RNA-seq (n=2 biological replicates). Gene set enrichment of direct NF-κB targets shows greater downregulation of NF-κB pathway in PBRM1-deficient cells compared to parental 786-O cells. P values were calculated by Kolmogorov-Smirnov test and q value was adjusted for multiple hypotheses testing using gene permutation. d) 786-O cells expressing dox inducible shRNA against ARID2 were treated with 1 μg/ml doxycycline for 2 days and the gene expression of NF-κB targets were quantified by RT-qPCR (n=3 technical replicates representative of 2 biological replicates). Data are presented as mean values+/−SD. * p<0.05, ** p<0.01 (two-sided t-test). e) Single cell clones of either PBRM1 or ARID2 knockout were created in 769-P cells. ARID2 loss led to depletion of other PBAF subunits including PBRM1 and BRD7 (Results representative of 2 biological replicates). f) Gene set enrichment of TNFα_signaling_via_NF-κB in 769-P cells expressing guide RNA against either non-targeting control (NTC), PBRM1 or ARID2 (n=3-4 biological replicates). P values were calculated by Kolmogorov-Smirnov test and q values were adjusted for multiple hypotheses testing using gene permutation. Immunoblotting (g) and colony formation (h) of 786-O cells expressing shRNA against empty vector (EV) or ARID2 (n=3 biological replicates). Data are presented as mean values+/−SD. * p<0.05. Immunoblotting (i) and colony formation (j) of 786-O cells expressing shRNA against empty vector (EV) or SMARCA4 (n=4 biological replicates). **** p<0.0001 (two-sided t-test).

FIG. 7 shows PBRM1 deficient PBAF complexes maintain RELA occupancy. a) Motif analysis reveals enrichment of TEAD and NF-κB motifs in gained SMARCA4 peaks after PBRM1 loss. b) Gene set enrichment of genes mapped to gained SMARCA4 peaks by ChIP-enrich (hypergeometric test). c) Differential RELA ChIP-seq peaks between parental and PBRM1-deficient 786-O cells were identified by Diffbind (n=3 biological replicates). Significantly altered regions are colored in pink (q<0.01). Numbers of significantly differential peaks (up and down) are shown. A positive log fold change refers to gain in PBRM1 KO over WT. d) Gained SMARCA4 and gained RELA peaks overlap significantly (hypergeometric test). e) RELA occupancy at gained and lost RELA peaks are shown next to SMARCA4 occupancy (black-WT; red-KO). f) UCSC browser view of TNF, a downstream target of NF-κB (black-WT; red-KO). g) 786-O cells were treated with SMARCA2/4 ATPase inhibitor BRM014 for 24 hours and ChIP-qPCR was performed to evaluate the amount of bound SMARCA4 and RELA at NF-κB target genes. h) Overlap of altered SMARCA4 or RELA peaks in parental 786-O or PBRM1-KO cells (hypergeometric test).

FIG. 8 shows that NF-κB inhibitor bortezomib preferentially targets PBRM1-deficient tumors. Colony formation of parental (WT) or PBRM1 knockout (KO) 786-O cells expressing shRNA against non-targeting control (NC), RELA, or NFKB1 cultured with a) 10% or b) 2% serum. c) Gene expression changes of NF-κB target genes in 786-O cells treated with 0.006 μM bortezomib (BTZ) or 0.6 UM Bay 11-7085 for 24 hours (n=3 technical replicates). * p-value<0.05 (two-sided t-test). Data are presented as mean values+/−SD. d) Gene expression changes of 786-O cells treated with DMSO control or 0.005 μM bortezomib for 9 hours measured by RNA-seq (n=3 biological replicates). e) Overlap between RELA targets and upregulated genes after PBRM1 KO (p=8.4E-101, hypergeometric test). Overlap between RELA targets and downregulated genes after bortezomib (BTZ) treatment (p=3.7E-55, hypergeometric test). f) Differential occupancy of RELA and SMARCA4 as revealed by ChIP-seq in 786-O PBRM1 WT and KO cells treated with DMSO or 0.005 UM bortezomib for 9 hours (n=2 biological replicates). Significantly altered regions are colored in pink (FDR<0.1 and fold change>1.5). Numbers of significantly differential peaks (up and down) are shown. A positive log fold change refers to gain in bortezomib treatment over DMSO. g) Colony formation of 786-O isogenic cells and h) NCC432 isogenic cells treated with bortezomib for a week. Tumor formation of isogenic 786-O cells (i) and NCC432 cells (j) treated with PBS control or 0.5 mg/kg bortezomib (BTZ) in NSG mice. (n=10 tumors per group for 786-O, and n=16 tumors for NCC432 PBS EV group and 12 tumors for NCC432 PBS PBRM1 group and 14 tumors for both NCC432 EV and PBRM1 BTZ groups). Data are presented as mean values+/−SEM. k) Model for the study.

FIG. 9 shows the effects of PBRM1 loss on tumor growth. a) Immunofluorescence staining validates the loss of PBRM1 by CRISPR mediated knockout in 786-O and HK2 cells (results representative of 2 biological replicates). b) Representative images of colony formation assay of isogenic pairs cultured in normal 10% FBS or low serum conditions (results representative of 3 biological replicates). c) PBRM1-deficient ccRCC cell lines were transduced with either empty vector (EV) control or wild-type PBRM1, cultured in soft agar for 3 weeks and assessed for colony formation potential. d) Shown are the photographs of 786-O and NCC432 tumors harvested at the end of the in vivo tumor formation studies. e) Tumor weights of NCC2112 xenograft tumors with and without PBRM1 were measured at the end of in vivo tumor formation studies (n=6 mice per group). Data are presented as mean values+/−SD. P-value was computed by two-tailed t-test.

FIG. 10 shows the effects of PBRM1 loss on PBAF integrity in NCC2112 cells. a) Co-immunoprecipitation of PBRM1 in NCC2112 cells expressing empty vector control or wildtype PBRM1 protein. b) Co-immunoprecipitation of SMARCA4 in NCC2112 cells expressing empty vector control or wildtype PBRM1. c) Co-immunoprecipitation of BRD7 and ARID2 in NCC2112 cells expressing empty vector control or PBRM1. Results representative of 2 biological replicates in a-c).

FIG. 11 shows genomic localization of PBRM1 and other PBAF subunits. a) PBRM1 ChIP-seq profiles of PBRM1-deficient NCC432 ccRCC cells expressing either empty vector (EV) or wild-type PBRM1. b) Genomic localization of PBRM1 binding sites in parental PBRM1-proficient cell lines (endogenous) and in PBRM1-deficient cell lines expressing wild-type PBRM1 cDNA (exogenous) annotated by ChIPseeker. c) Genomic localization of PBRM1, BRD7 and ARID2 ChIP-seq in 786-O cells shows promoter-centric occupancy of PBRM1 and BRD7 but equal distribution of ARID2 in both promoters and distal regions. d) While the majority of the PBRM1 binding sites are bound by all subunits of the PBAF subunits and SMARCA4 (canonical sites), there are also sites that are bound by PBRM1- or ARID2-deficient PBAF subcomplexes.

FIG. 12 shows the effects of PBRM1 loss on the genomic occupancy of PBAF subunits. a) Altered SMARCA4 peaks were linked to promoters of the nearest genes by ChIP-Enrich (defined as +2.5 kb of TSS) and the log 2 fold change (left) or difference in gene expression (right) after PBRM1 KO in 786-O cells (n=1527 genes mapped to gained regions and n=134 genes mapped to lost regions). P-values were computed by two-sided Wilcoxon test. Boxplots presented as median values+/−25%. Lower whisker is smallest observation greater than or equal to 25% quantile-1.5× interquantile range (IQR). Upper whisker represents the largest observation less than or equal to 75%+1.5×IQR. b) Peak regions of all the PBAF subunits were merged, and differences in occupancy of the various PBAF subunits between parental (WT) and PBRM1 knockout (KO) 786-O cells are plotted against the differences in PBRM1 occupancy for all regions. Many regions with large changes in SMARCA4 and ARID2 occupancy have no corresponding changes in PBRM1 occupancy, suggesting that these regions are located away from PBRM1 binding sites. Shown are the spearman correlation coefficients of the changes in the occupancy of PBAF subunits after PBRM1 KO. c) Spearman correlation of changes in chromatin occupancy between parental (WT) and PBRM1 knockout (KO) 786-O cells for all PBAF bound regions.

FIG. 13 shows that PBRM1 loss is associated with gain in NF-κB activity across various models. a) BETA analysis with P-values calculated by Kolmogorov-Smirnov test. b) Gene expression changes of selected NF-κB targets after PBRM1 expression in 786-O PBRM1 KO cells. c-h) Enrichment of TNFα_signaling_via_NF-κB geneset in c) 786-O PBRM1 KO cells expressing empty vector or PBRM1, d) 769-P cells expressing sgRNA against non-targeting control or PBRM1), e) PBRM1-deficient NCC432 and f) PBRM1-deficient A704 cells expressing empty vector or PBRM1, g) genetic mouse model with VHL single knockout or double knockout of PBRM1 and VHL (DKO) and h) TCGA data (KIRC) of tumors deficient or proficient in PBRM1. P-values were calculated by Kolmogorov-Smirnov test and q value was adjusted for multiple hypotheses testing using gene permutation. i) Heatmap shows all the significantly upregulated genes of the TNFα_signaling_via_NF-κB geneset in the PBRM1-deficient TCGA KIRC samples relative to PBRM1-proficient samples. P-value computed by Wald test. j) Normalized gene expression of selected upregulated NF-κB targets in NCC432 expressing empty vector control or wildtype PBRM1 after TNFα stimulation for 6 hours measured by RT-qPCR (n=3 technical replicates representative of 2 biological replicates). Data are presented as mean values+/−SD. * p<0.05, ** p<0.01 (two-sided t-test). k-m) Enrichment of the Hallmark geneset inflammatory response in k) 786-O PBRM1 KO cells expressing empty vector or PBRM1, 1) PBRM1-deficient NCC432 cells expressing empty vector or PBRM1 and m) genetic mouse model with VHL single knockout or double knockout of PBRM1 and VHL (DKO). P values were calculated by Kolmogorov-Smirnov test and q value was adjusted for multiple hypotheses testing using gene permutation. n) Gene expression changes of NF-κB target genes after shRNA-mediated knockdown of NF-κB subunits measured by RT-qPCR (n=3 technical replicates). * p<0.05, ** p<0.01, *** p<0.001, **** p<0.0001 (two-sided t-test). Data are presented as mean values+/−SD. o) Overlap of RELA targets defined by RELA ChIP occupancy and decreased expression after shRELA (p<0.05 and log 2FC<−0.5) with downregulated genes after PBRM1 restoration in PBRM1-KO 786-O cells (p<0.05 and |log 2FC|>0.3). P value of the overlap was computed by hypergeometric test.

FIG. 14 shows that PBRM1-deficient tumors depend on SMARCA4 and ARID2 for NF-κB activity and tumor formation. a) NCC432 cells expressing either empty vector or PBRM1 were treated with SMARCA2/4 ATPase inhibitor BRM014 for 3 days and the expression of NF-κB targets was evaluated by RT-qPCR (n=3 technical replicates representative of 2 biological replicates). Data are presented as mean values+/−SD. b) Colony formation of 786-O cells expressing shRNA against non-targeting control (NTC) or ARID2. c) Colony formation of 786-O cells expressing shRNA against non-targeting control (NTC) or SMARCA4. d) Immunoblotting and e) colony formation of NCC432 cells expressing shRNA against non-targeting control (NTC) or ARID2 (n=3 biological replicates). Data are presented as mean values+/−SD. * p<0.05, ** p<0.01 (two-sided t-test). f) Immunoblotting and g) colony formation of NCC432 cells expressing shRNA against non-targeting control (NTC) or SMARCA4 (n=4 biological replicates). Data are presented as mean values+/−SD. *** p<0.001, **** p<0.0001 (two-sided t-test). In vivo tumor formation in h) 786-O parental cells and i) 786-0 PBRM1 KO cells expressing shRNA against non-targeting control (NTC), ARID2 (clone 1) or SMARCA4 (clone 2) (n=10 tumors per group). Data are presented as mean values+/−SEM for tumor growth and mean values+/−SD for tumor weight. **** p<0.0001 (two-sided t-test).

FIG. 15 shows that NF-κB activation is mediated by changes in the chromatin. a) Geneset enrichment of genes mapped to gained ARID2 and gained BRD7 peaks by ChIP-enrich (hypergeometric test). b) Protein expression of NF-κB subunits in cytoplasmic (cyto), soluble nuclear (sol nuc) or chromatin fractions in 786-O PBRM1 WT, PBRM1 KO, PBRM1 KO expressing empty vector or PBRM1 (representative blot of 2 biological replicates). c) RELA peaks overlap significantly with total and gained ARID2 peaks (hypergeometric test). d) UCSC browser view of selected NF-κB target genes.

FIG. 16 shows that bortezomib abrogates RELA occupancy and selectively targets PBRM1 deficiency. a) Colony formation of 786-O cells with and without PBRM1 treated with DMSO or NF-κB inhibitor, Bay 11-7085. b) Single sample gene-set enrichment (ssGSEA) of TNFα_signaling_via_NF-κB in 786-O PBRM1 WT or KO cells treated with DMSO or 0.005 μM bortezomib (n=3 biological replicates). Data are presented as median+/−25%. Lower whisker is smallest observation greater than or equal to 25% quantile-1.5*interquantile range (IQR). Upper whisker represents the largest observation less than or equal to 75%+1.5*IQR. Amount of normalized RELA protein in the c) input or d) co-immunoprecipitated with SMARCA4 from 3 independent runs. Data are presented as mean values+/−SD. e) Tornado plots of RELA and SMARCA4 ChIP-seq signals in parental 786-0 or PBRM1-KO 786-O cells treated with DMSO or 0.005 μM bortezomib for 9 hours. f) Kaplan-Meier curves of NSG mice bearing 786-O isogenic cells treated with PBRM1 control or 0.5 mg/kg bortezomib (WT: n=10 PBS arm, median survival=39 days; n=10 bortezomib arm, median survival=52 days, HR=4.1, p=3E-6, log rank test; KO: n=10 PBS arm, median survival=35 days, n=10 bortezomib arm, median survival=66 days, HR=4.1, p=3E-6, log rank test). g) Kaplan-Meier curves of NSG mice bearing NCC432 isogenic cells treated with PBRM1 control or 0.5 mg/kg bortezomib (EV: n=16 PBS arm, median survival=42 days; n=14 bortezomib arm, median survival=49 days, HR=2.5, p=0.0009, log rank test; PBRM1: n=12 PBS arm, median survival=55 days; n=14 bortezomib arm, median survival=58 days, HR=1.2, p=0.5, log rank test). h) 786-O PBRM1 WT or KO cells were screened against the high-specific SGC epigenetic probe collection and cell viability was measured by colony formation after 1 week of treatment relative to DMSO control.

### DETAILED DESCRIPTION

The current invention is predicated on the discovery that Nuclear factor kappa-light-chain-enhancer of activated B cells (NF-κB) is a major protein activated upon PB1 loss in cancer. Without being bound by theory, NF-κB promotes all aspects of cancer hallmarks, including enhancing tumor proliferation, epithelial-mesenchymal transition, angiogenesis and circumventing apoptosis. Cancer cell-intrinsic inflammation mediated by NF-κB drives ccRCC metastasis. Furthermore, since NF-κB is a critical mediator of inflammatory responses, NF-κB activation by PB1 loss may play a direct role in activating other immune-related pathways such as interferon gamma response and IL-6 JAK-STAT3 signaling.

Accordingly, the present invention relates to a method of identifying a cancer that is likely to be responsive to treatment with an inhibitor of NF-κB signalling in a subject. The method may comprise detecting an indicator that is indicative of a level or activity of Protein polybromo-1 (PB1) in a sample obtained from the subject. The cancer may be identified as being likely to be responsive to the inhibitor of NF-κB signalling when the level or activity of PB1 indicated by the indicator corresponds to an absence or decreased level or activity of PB1 in the sample as compared to a control.

Disclosed herein is a method of identifying a cancer that is likely to be responsive to treatment with an inhibitor of Nuclear factor kappa-light-chain-enhancer of activated B cells (NF-κB) signalling in a subject, the method comprising detecting an indicator that is indicative of a level or activity of Protein polybromo-1 (PB1) in a sample obtained from the subject, wherein the cancer is identified as being likely to be responsive to the inhibitor of NF-κB signalling when the level or activity of PB1 indicated by the indicator corresponds to an absence or decreased level or activity of PB1 in the sample as compared to a control.

As used herein, “PB1” or “polybromo-1” refers to the protein encoded by the PBRM1 gene, and all fragments, isoforms, variants (e.g., allelic and splice variants) and derivatives thereof. The term “PB1” refers to the human protein (with amino acid sequence given in SEQ ID NO: 1) and its orthologs (e.g., mouse PB1). Also encompassed are polypeptides comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, 99.5% or more sequence identity with SEQ ID NO: 1, or a portion thereof. Such polypeptides can have a function of the full-length PB1 protein, or exhibit reduced or eliminated PBRM1 function (e.g., due to the presence of non-synonymous or truncating mutations). Human PB1 is listed as Gene ID 55193 on the United States National Center for Biotechnology Information database.

As used herein, “VHL” or “Von Hippel-Lindau” refers to the protein encoded by the VHL gene, and all fragments, isoforms, variants (e.g., allelic or splice variants) and derivatives thereof. The term “VHL” refers to the human protein (with amino acid sequence given in SEQ ID NO: 2) and its orthologs (e.g., mouse VHL). Also encompassed are polypeptides comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, 99.5% or more sequence identity with SEQ ID NO: 2, or a portion thereof. Such polypeptides can have a function of the full-length VHL protein, or exhibit reduced or eliminated VHL function (e.g., due to the presence of non-synonymous or truncating mutations). Human VHL is listed as Gene ID 7428 on the United States National Center for Biotechnology Information database.

### Human PB1 Amino Acid Sequence

(SEQ ID NO: 1)
MGSKRRRATSPSSSVSGDFDDGHHSVSTPGPSRKRRRLSNLPTVDPIAVC
HELYNTIRDYKDEQGRLLCELFIRAPKRRNQPDYYEVVSQPIDLMKIQQK
LKMEEYDDVNLLTADFQLLFNNAKSYYKPDSPEYKAACKLWDLYLRTRNE
FVQKGEADDEDDDEDGQDNQGTVTEGSSPAYLKEILEQLLEAIVVATNPS
GRLISELFQKLPSKVQYPDYYAIIKEPIDLKTIAQRIQNGSYKSIHAMAK
DIDLLAKNAKTYNEPGSQVFKDANSIKKIFYMKKAEIEHHEMAKSSLRMR
TPSNLAAARLTGPSHSKGSLGEERNPTSKYYRNKRAVQGGRLSAITMALQ
YGSESEEDAALAAARYEEGESEAESITSFMDVSNPFYQLYDTVRSCRNNQ
GQLIAEPFYHLPSKKKYPDYYQQIKMPISLQQIRTKLKNQEYETLDHLEC
DLNLMFENAKRYNVPNSAIYKRVLKLQQVMQAKKKELARRDDIEDGDSMI
SSATSDTGSAKRKSKKNIRKQRMKILFNVVLEAREPGSGRRLCDLFMVKP
SKKDYPDYYKIILEPMDLKIIEHNIRNDKYAGEEGMIEDMKLMFRNARHY
NEEGSQVYNDAHILEKLLKEKRKELGPLPDDDDMASPKLKLSRKSGISPK
KSKYMTPMQQKLNEVYEAVKNYTDKRGRRLSAIFLRLPSRSELPDYYLTI
KKPMDMEKIRSHMMANKYQDIDSMVEDFVMMENNACTYNEPESLIYKDAL
VLHKVLLETRRDLEGDEDSHVPNVTLLIQELIHNLFVSVMSHQDDEGRCY
SDSLAEIPAVDPNFPNKPPLTFDIIRKNVENNRYRRLDLFQEHMFEVLER
ARRMNRTDSEIYEDAVELQQFFIKIRDELCKNGEILLSPALSYTTKHLHN
DVEKERKEKLPKEIEEDKLKREEEKREAEKSEDSSGAAGLSGLHRTYSQD
CSFKNSMYHVGDYVYVEPAEANLQPHIVCIERLWEDSAGEKWLYGCWFYR
PNETFHLATRKFLEKEVFKSDYYNKVPVSKILGKCVVMFVKEYFKLCPEN
FRDEDVFVCESRYSAKTKSFKKIKLWTMPISSVRFVPRDVPLPVVRVASV
FANADKGDDEKNTDNSEDSRAEDNFNLEKEKEDVPVEMSNGEPGCHYFEQ
LHYNDMWLKVGDCVFIKSHGLVRPRVGRIEKVWVRDGAAYFYGPIFIHPE
ETEHEPTKMFYKKEVFLSNLEETCPMTCILGKCAVLSFKDFLSCRPTEIP
ENDILLCESRYNESDKQMKKFKGLKRFSLSAKVVDDEIYYFRKPIVPQKE
PSPLLEKKIQLLEAKFAELEGGDDDIEEMGEEDSEVIEPPSLPQLQTPLA
SELDLMPYTPPQSTPKSAKGSAKKEGSKRKINMSGYILFSSEMRAVIKAQ
HPDYSFGELSRLVGTEWRNLETAKKAEYEERAAKVAEQQERERAAQQQQP
SASPRAGTPVGALMGVVPPPTPMGMLNQQLTPVAGMMGGYPPGLPPLQGP
VDGLVSMGSMQPLHPGGPPPHHLPPGVPGLPGIPPPGVMNQGVAPMVGTP
APGGSPYGQQVGVLGPPGQQAPPPYPGPHPAGPPVIQQPTTPMFVAPPPK
TQRLLHSEAYLKYIEGLSAESNSISKWDQTLAARRRDVHLSKEQESRLPS
HWLKSKGAHTTMADALWRLRDLMLRDTLNIRQAYNLENV

### Human VHL Amino Acid Sequence

(SEQ ID NO: 2)
MPRRAENWDEAEVGAEEAGVEEYGPEEDGGEESGAEESGPEESGPEELGA
EEEMEAGRPRPVLRSVNSREPSQVIFCNRSPRVVLPVWLNFDGEPQPYPT
LPPGTGRRIHSYRGHLWLFRDAGTHDGLLVNQTELFVPSLNVDGQPIFAN
ITLPVYTLKERCLQVVRSLVKPENYRRLDIVRSLYEDLEDHPNVQKDLER
LTQERIAHQRMGD

The term “tumor,” as used herein, refers to any neoplastic cell growth and proliferation, whether malignant or benign, and all pre-cancerous and cancerous cells and tissues. The terms “cancer” and “cancerous” refer to or describe the physiological condition in mammals that is typically characterized in part by unregulated cell growth. As used herein, the term “cancer” refers to non-metastatic and metastatic cancers, including early stage and late stage cancers. The term “precancerous” refers to a condition or a growth that typically precedes or develops into a cancer. By “non-metastatic” is meant a cancer that is benign or that remains at the primary site and has not penetrated into the lymphatic or blood vessel system or to tissues other than the primary site. Generally, a non-metastatic cancer is any cancer that is a Stage 0, I, or II cancer, and occasionally a Stage III cancer. By “early stage cancer” is meant a cancer that is not invasive or metastatic or is classified as a Stage 0, I, or II cancer. The term “late stage cancer” generally refers to a Stage III or Stage IV cancer, but can also refer to a Stage II cancer or a substage of a Stage II cancer. One skilled in the art will appreciate that the classification of a Stage II cancer as either an early stage cancer or a late stage cancer depends on the particular type of cancer. Illustrative examples of cancer include, but are not limited to, carcinoma, lymphoma, blastoma, sarcoma, and leukemia or lymphoid malignancies. More particular examples of such cancers include, but are not limited to, glioma, breast cancer, prostate cancer, ovarian cancer, cervical cancer, pancreatic cancer, colorectal cancer, lung cancer, hepatocellular cancer, gastric cancer, liver cancer, bladder cancer, cancer of the urinary tract, thyroid cancer, renal cancer, melanoma, brain cancer, non-small cell lung cancer, squamous cell cancer of the head and neck, endometrial cancer, multiple myeloma, rectal cancer, and esophageal cancer.

In some embodiments the cancer is a renal cancer, such as renal cell cancer, transitional cell cancer or Wilms tumor (i.e., nephroblastoma).

In some embodiments, the renal cancer is renal cell carcinoma (RCC). The different types of RCC are generally distinguished by the way that cancer cells appear when viewed under a microscope, such as clear cell RCC (ccRCC), papillary RCC, chromophobe RCC, collecting duct RCC, and other unclassified RCC. In one embodiment the renal cancer is clear cell renal cell carcinoma (ccRCC).

An “indicator” as referred to herein may be a transcript level or protein level of a gene, or a variation in the copy number or sequence of a gene or operably connected transcriptional control region or UTR. The indicator may be detected using techniques that are well known in the art.

Methods of detecting protein level include, but are not limited to, immunohistochemistry (IHC), immunofluorescence, Western blot analysis, dot blotting, immunoprecipitation, radioimmunoassay, molecular binding assays, ELISA, ELIFA, fluorescence activated cell sorting (“FACS”), surface plasmon resonance (SPR), high performance liquid chromatography (HPLC), optical spectroscopy, mass spectrometry, proteomics, quantitative blood-based assays (as for example serum ELISA) and/or biochemical enzymatic activity assays.

Methods of detecting gene or transcript level include, but are not limited to, in situ hybridization, fluorescence in situ hybridization (FISH), Southern analysis, Northern analysis, targeted gene sequencing, whole genome sequencing, polymerase chain reaction (PCR) including quantitative real time PCR (qRT-PCR), multiplex qPCR, reverse transcription polymerase chain reaction (RT-PCR) and other amplification-type detection methods, such as, for example, branched DNA, SISBA, TMA and the like, RNAseq, microarray analysis, MassARRAY, serial analysis of gene expression (SAGE™), gene expression profiling, as well as any one of the wide variety of assays that can be performed by protein, gene, and/or tissue array analysis. Detection may be performed on a chip or microarray, e.g., a nucleic acid or protein microarray.

Protein activity may be detected by quantitative or qualitative analysis of protein binding to a natural or engineered substrate, ligand, antigen or other molecular target, using methods known in the art. For example, PB1 activity may be detected by detecting PB1 binding to DNA or chromatin using DNA or chromatin immunoprecipitation, and VHL activity may be detected by detecting VHL binding to natural or synthetic ligands, e.g., the fluorescently-labelled ligand BODIPY FL VH032.

DNA, RNA or protein may be isolated from a sample using methods known in the art or using commercially available isolation kits. RNA samples can be further enriched in particular species before analysis, e.g., mRNA can be further purified from an RNA sample using poly-T oligonucleotides that hybridize with the poly-A tails on mRNA.

The level or activity of PB1 as indicated by the indicator in the sample and in the control may first be normalized before comparison. Normalization can be used to control for unwanted biological variation. Normalization can be performed using methods known in the art. In a non-limiting example, normalization can be achieved by dividing the measured expression level of PB1 (in the sample or control) by the measured expression level of a reference or housekeeping gene. Useful reference genes are genes that show a low variation in their expression level across a variety of different samples and subjects. For example, a useful reference gene will show the same expression level in samples derived from subjects who have cancer and in samples derived from subjects who do not have cancer. The variation in expression level can be quantified by different methods known in the art. For example, the variation in expression level of a gene can be quantified by calculating the coefficient of variation in the expression level of a particular gene across a set of different samples.

The activity of PB1 as indicated by the indicator in the sample and in the control may be normalized before comparison. In a non-limiting example, normalization for activity can be achieved by dividing the measured activity level of PB1 protein (in the sample or control) by the PB1 protein concentration.

The “control” as referred to herein may be one or more non-cancerous samples taken from the same subject (e.g. from a healthy tissue, or from an archived or stored sample before the onset of cancer in the subject), or one or more non-cancerous samples taken from another subject (e.g. a healthy subject who does not suffer from cancer), or one or more non-cancerous samples taken from a population of subjects (e.g. a population of healthy subjects who do not have cancer), or one or more non-cancerous samples taken from a cell or tissue depository. The control may also be a pre-determined value or an average value, e.g., a value that represents the average PB1 gene expression level in a population of healthy subjects, normalized to the average expression level of a housekeeping gene in the same population.

The method may comprise comparing the level or activity of PB1 or VHL, or a value representative of the level or activity of PB1 or VHL, to a control. The comparison may be carried out manually or it may be automated, e.g., by relying on a computer algorithm with the relevant values as inputs.

As used herein, the term “decrease” or “decreased’ with reference to an indicator level or activity refers to a statistically significant and measurable decrease in the indicator level or activity as compared to a control. The decrease in the level or activity of PB1 in the sample that is indicative of responsiveness to an inhibitor of NF-κB signalling may be a decrease of at least 75%, at least 80%, at least 85%, at least 90%, or at least 95% compared to the control. The decrease in the level or activity of PB1 in the sample that is indicative of responsiveness to an inhibitor of NF-κB signalling may be a decrease of about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%.

An absence or decreased level or activity of a protein may be the result of, for example, decreased expression from the encoding gene, increased degradation of the mRNA transcript or protein, or loss-of-function mutations that alter the structure of the protein or the interaction of the protein with other proteins in the same protein complex or involved in the same or a different pathway. The structure of the protein may be altered by mutations (e.g., non-synonymous substitutions, additions, deletions) which change the amino acid sequence of the protein. A “loss-of-function mutation” herein refers to any nucleic acid mutation which results in reduced or eliminated protein levels and/or function. Non-limiting examples of such mutations include non-synonymous base substitutions, base insertions, base deletions, frameshift mutations, nonsense mutations, splice-site mutations, epigenetic modifications (e.g., methylation, phosphorylation, acetylation, ubiquitylation, sumoylation, histone acetylation, histone deacetylation, and the like), and combinations thereof. Such mutations reduce or eliminate protein levels and/or function by eliminating proper coding sequences required for proper protein translation and/or coding for proteins that are non-functional or have reduced function (e.g., deletion of functional and/or structural domains, reduction in protein stability, alteration of sub-cellular localization, and the like).

Loss-of-function mutations in the human PBRM1 gene may include A403G, T724C, G856T, 1373delA (deletion at position 1373), 1983delA (deletion at position 1983), C2128T, G2380T, A2392T, T2887C, G3394A, G3691T, A3743G, G3859A, G4339C, 4370insTT (insertion at position 4370), wherein the numbers refer to the nucleotide position in the human PBRM1 gene. Other PBRM1 loss-of-function mutations are searchable on the NCBI Clin Var database (https://www.ncbi.nlm.nih.gov/clinvar), non-limiting examples of which include NM_001405607.1 (PBRM1):c.4792C>T (p.Pro1598Ser), NM_001405607.1 (PBRM1):c.4043_4050del (p.Asp1348fs), and chromosome 3 deletions like GRCh38/hg38 3p21.31-14.3 (chr3:49461000-55314500)x1, GRCh38/hg38 3p21.2-14.3 (chr3:51394434-55064449)x1, GRCh38/hg38 3p21.2-14.3 (chr3:51394434-55064449)x1, NC_000003.11:g.(?_52109903)_(53164416_?)del, NC_000003.11:g.(?_52018081)_(53845433_?)del, GRCh37/hg19 3p21.2-21.1 (chr3:52195134-52869037)x1, and GRCh37/hg19 3p21.2-14.2 (chr3:52086599-59689209)x1.

Loss-of-function mutations in the human VHL gene may include G154T, C217T, 223insT (insertion at position 223), G361C, G445T, 446delG (deletion at position 446), A479T, 472insTTT (insertion at position 472), 589delG (deletion at position 589). Other VHL loss-of-function mutations are searchable on the NCBI ClinVar database (https://www.ncbi.nlm.nih.gov/clinvar), non-limiting examples of which include pathogenic mutations [e.g., GRCh38/hg38 3p26.3-25.3 (chr3:20213-11221602)x1, NC_000003.11:g.10182332_10190098del, and NC_000003.11:g.10178710_10187165del], likely pathogenic mutations [e.g., NM_000551.4 (VHL):c.340+770T>C, NM_000551.4 (VHL):c.397A>C (p.Thr133Pro), and NM_000551.4 (VHL):c.547del (p.Ser183fs)], and other mutations [e.g., NM_000551.3 (VHL):c.-118C>T and NM_000551.4 (VHL):c.-15_4delinsCGGCCCGGGTGGTCTGGTCTCCG (p.Met1fs)].

Certain mutations may give rise to the simultaneous loss of function of both PB1 and VHL, e.g., the loss of chromosome arm 3p in humans.

As used herein, the term “responsiveness” refers to the likeliness of a cancer, a cancer cell or a cancer subject responding to an inhibitor of NF-κB signaling, i.e. showing decreased proliferation/growth and/or increased cell death after being treated with the inhibitor. In some embodiments, the responsiveness can be scaled as insensitive (i.e., less likely to respond) or sensitive (likely to respond).

In one embodiment, Von Hippel-Lindau (VHL) is absent in the cancer or the cancer has a decreased expression or activity of Von Hippel-Lindau (VHL).

The method may further comprise detecting an indicator that is indicative of a level or activity of VHL in a sample obtained from the subject, for example, VHL transcript or protein. The same methods for detecting the level or activity of PB1 may be used to detect the level or activity of VHL. The cancer may be identified as being likely to be responsive to the inhibitor of NF-κB signalling when the level or activity of both PB1 and VHL as indicated by their respective indicators correspond to an absence or decreased level or activity of PB1 and VHL in the sample as compared to the control.

The term “inhibitor” as used herein refers to an agent that decreases or inhibits at least one function or biological activity of a target molecule or signalling pathway. An inhibitor may be any therapeutic or preventative agent. The term “inhibitor” is not to be construed narrowly but extends to small molecules, proteinaceous molecules such as peptides, proteins (e.g., antibodies, enzymes, PROTACs, etc.) and protein fragments, nucleic acid molecules such as DNA (e.g., aptamers), RNA (e.g., mRNA, short hairpin RNA, small interfering RNA, etc.) and mimetics and chemical analogs thereof, and combinations of the above (e.g., antibody-drug conjugates). The term “inhibitor” also extends to nucleic acid constructs including vectors such as viral or non-viral vectors, expression vectors and plasmids for expression in a range of cells.

In one embodiment, the inhibitor is an inhibitor of NF-κB signalling. NF-κB signalling pathways are known in the art and include both the canonical and non-canonical pathways. The inhibitor may act on NF-κB or on one or more protein components of an NF-κB signaling pathway. The inhibitor may decrease or eliminate the level or activity of NF-κB, or increase, decrease or eliminate the level or activity of one or more protein components of an NF-κB signaling pathway. Examples of protein components of NF-κB signaling pathways include but are not limited to BTK, LYN, BCR Igα, BCR Igβ, Syk, Blnk, PLCγ2, PKCβ, DAG, CARMA1, BCL10, MALT1, PI3K, PIP3, AKT, p38 MAPK, ERK, COT, IKKα, IKKβ, IKKγ, IKKε, NIK, RelA/p65, P105/p50, c-Rel, RelB, p52, NIK, Leu13, CD81, CD19, CD21 and its ligands in the complement and coagulation cascade, TRAF6, ubiquitin ligase, Tab2, TAK1, TBK1, NEMO, NOD2, RIP2, Lck, fyn, Zap70, LAT, GRB2, SOS, CD3 zeta, Slp-76, GADS, ITK, PLCy1, PKCO, ICOS, CD28, SHP2, SAP, SLAM and 2B4.

Non-limiting examples of inhibitors of NF-κB signalling include BTK inhibitors (e.g., Zanubrutinib, Acalabrutinib, Ibrutinib, Zanubrutinib, Dasatinib, Orelabrutinib, Tirabrutinib, Rilzabrutinib, Evobrutinib, ABBV-105, Dasatinib, SAR-442168, Branebrutinib, Branebrutinib, Remibrutinib, TAS-5315, BMS-986142, Fenebrutinib, Poseltinib and Spebrutinib); IRAK inhibitors (e.g., Pacritinib, PF-06650833, CA-4948, R-835, BAY-1834845 and BAY-1830839); c-IAP inhibitors (e.g., Birinapant, APG-1387, LCL-161, ASTX660, Debio 1143, CUDC-427 and AT406); CD30 inhibitors (e.g., Brentuximab); PI3K inhibitors (e.g., Copansilib, Idelalisib); nuclear export inhibitors (e.g., Leptomycin B, CBS9106, Selinexor, Verdinexor, Eltanexor, Felezonexor), nuclear translocation inhibitors (e.g., Iguratimod); Histone deacetylase inhibitors (e.g., Vorinostat, Romidepsin, Belinostat, Panobinostat, Tucidinostat); DNA acetylation inhibitors (e.g., Azacitidine, Decitabine) and polyphenols (e.g., curcumin, capsaicin, apigenin, oleandrin, quercetin, resveratrol, cinnamaldehyde and epigallocatechin-3-gallate). Non-exhaustive lists of NF-κB signalling inhibitors may also be found in Tables 1-3 of WO2008043157, and the table in WO2020118159A1.

In one embodiment, the inhibitor of NF-κB signalling is a proteasome inhibitor. The proteasome inhibitor may inhibit one or both of the 19S and 20S subunits of the 26S proteasome. Examples of proteasome inhibitors include, but are not limited to, bortezomib, ixazomib, marizomib, carfilzomib, oprozomib, disulfiram, epigallocatechin-3-gallate, salinosporamid A, ONX0912, CEP-18770, Epoxomicin, FK506, MG101, MG115 and MG132. In one embodiment, the inhibitor is bortezomib or carfilzomib.

The terms “patient,” “subject,” “host” or “individual” used interchangeably herein, refer to any subject, particularly a vertebrate subject, and even more particularly a mammalian subject, for whom therapy or prophylaxis is desired. Suitable vertebrate animals that fall within the scope of the invention include, but are not restricted to, any member of the subphylum Chordata including primates (e.g., humans, monkeys and apes), and includes species of monkeys such as from the genus Macaca (e.g., cynomologus monkeys such as Macaca fascicularis, and/or rhesus monkeys (Macaca mulatta)) and baboon (Papio ursinus), as well as marmosets (species from the genus Callithrix), squirrel monkeys (species from the genus Saimiri) and tamarins (species from the genus Saguinus), as well as species of apes such as chimpanzees (Pan troglodytes), rodents (e.g., mice rats, guinea pigs), lagomorphs (e.g., rabbits, hares), bovines (e.g., cattle), ovines (e.g., sheep), caprines (e.g., goats), porcines (e.g., pigs), equines (e.g., horses), canines (e.g., dogs), felines (e.g., cats), avians (e.g., chickens, turkeys, ducks, geese, companion birds such as canaries, budgerigars etc.), marine mammals (e.g., dolphins, whales), reptiles (snakes, frogs, lizards etc.), and fish. In specific embodiments, the subject is a primate such as a human. However, it will be understood that the aforementioned terms do not imply that symptoms are present.

A sample can be a biological sample which refers to the fact that it is derived or obtained from a living organism. The sample may be obtained from a whole organism or from an in vitro culture (e.g., cells or organs grown in culture). A “biological sample” also refers to a cell or population of cells or a quantity of tissue or fluid from a subject. Most often, a sample has been removed from a subject, but the term “biological sample” can also refer to cells or tissue analyzed in vivo, i.e., without removal from the subject. Often, a “biological sample” will contain cells from a subject, but the term can also refer to non-cellular biological material, such as non-cellular fractions of blood, saliva, or urine. The biological sample may be from a resection, bronchoscopic biopsy, or core needle biopsy of a primary, secondary or metastatic tumor, or a cellblock from pleural fluid. In addition, fine needle aspirate biological samples are also useful. In one embodiment, a biological sample is ascites. Biological samples also include explants and primary and/or transformed cell cultures derived from patient tissues. A biological sample can be provided by removing a sample of cells from subject, but can also be accomplished by using previously isolated cells or cellular extracts (e.g. isolated by another person, at another time, and/or for another purpose). Archival tissues, such as those having treatment or outcome history may also be used. Biological samples include, but are not limited to, tissue biopsies, scrapes (e.g. buccal scrapes), whole blood, plasma, serum, urine, saliva, cell culture, or cerebrospinal fluid.

In one embodiment, the sample is a tumor sample. The tumor sample may be a cancer sample or blood sample.

The term “tumor sample” as used herein means a sample comprising tumor material obtained from a cancerous patient. The term encompasses clinical samples, for example tissue obtained by surgical resection and tissue obtained by biopsy, such as for example a core biopsy or a fine needle biopsy. The term also encompasses samples comprising tumor cells obtained from sites other than the primary tumor, e.g., circulating tumor cells, as well as well as preserved tumor samples, such as formalin-fixed, paraffin-embedded tumor samples or frozen tumor samples. The term encompasses cells that are the progeny of the patient's tumor cells, e.g., cell culture samples derived from primary tumor cells or circulating tumor cells. The term encompasses samples that may comprise protein or nucleic acid material shed from tumor cells in vivo, e.g., bone marrow, blood, plasma, serum, and the like. The term also encompasses samples that have been enriched for tumor cells or otherwise manipulated after their procurement and samples comprising polynucleotides and/or polypeptides that are obtained from a patient's tumor material.

As used herein, “obtained” is meant to come into possession. Obtaining a sample, such as a biological sample, can include coming into the possession of a sample that has already been taken from a subject, as well as actively taking a sample from a subject.

The sample may be used directly as obtained from the subject or following a pretreatment to modify the character of the sample. For example, such pretreatment may include preparing plasma from blood, diluting viscous fluids and so forth. Methods of pretreatment may also involve, but are not limited to, filtration, precipitation, dilution, distillation, mixing, centrifugation, freezing, lyophilisation, concentration, c amplification, nucleic acid fragmentation, inactivation of interfering components, the addition of reagents, cell lysis, etc. If such methods of pretreatment are employed with respect to the sample, such pretreatment methods are typically such that the indicator to be detected (e.g., PB1 transcript, PB1 protein or PB1-encoding nucleic acid) remain in the sample, sometimes at a concentration proportional to that in an untreated sample (i.e., a sample that is not subjected to any such pretreatment). A sample expressly encompasses fractions or processed portions thereof.

This present invention also provides a method of treating a cancer in a subject, the method comprising a) detecting an indicator that is indicative of a level or activity of PB1 in a sample obtained from the subject, wherein the cancer is identified as being likely to be responsive to the inhibitor of NF-κB signalling when the level or activity of PB1 indicated by the indicator corresponds to an absence or decreased level or activity of PB1 in the sample as compared to the control; and b) administering an inhibitor of NF-κB signalling to the subject when the subject is found likely to be responsive to treatment with the inhibitor of NF-κB signalling. In one embodiment, Von Hippel-Lindau (VHL) is absent in the cancer or the cancer has a decreased expression or activity of Von Hippel-Lindau (VHL).

The inhibitor may be administered in combination with other therapeutic interventions (e.g., chemotherapy, radiation therapy, photodynamic therapy, radiofrequency ablation, or other pharmacological interventions such as anti-angiogenic therapy) for cancer treatment. The method may further comprise monitoring the cancer cell following the contact with the inhibitor.

As used herein, the terms “treatment,” “treating,” and the like, refer to obtaining a desired pharmacologic and/or physiologic effect. The effect may be therapeutic in terms of a partial or complete cure for a disease or condition (e.g., a hematologic malignancy) and/or adverse effect attributable to the disease or condition. These terms also cover any treatment of a condition or disease in a mammal, particularly in a human, and include: (a) inhibiting the disease or condition, i.e., arresting its development; or (b) relieving the disease or condition, i.e., causing regression of the disease or condition.

An effective amount of the inhibitor may be administered to the subject. The term “effective amount” or “pharmaceutically effective amount” as used herein refers to an amount or quantity of a drug or pharmaceutically active substance which is sufficient to elicit the required or desired therapeutic response, or an amount which is sufficient to elicit an appreciable biological response when administered to a subject. The effective amount will vary depending upon the health and physical condition of the subject to be treated, the taxonomic group of subject to be treated, the formulation of the composition, the assessment of the medical situation, and other relevant factors. It is expected that the amount will fall in a relatively broad range that can be determined through routine trials. The inhibitor may be administered in combination one or more cancer therapies.

Disclosed herein is a method of inhibiting proliferation of a cancer cell, the method comprising contacting the cancer cell with an inhibitor of NF-κB signalling, wherein PB1 is absent in the cancer cell or wherein the cancer cell has a decreased level or activity of PB1 as compared to a control. In one embodiment, Von Hippel-Lindau (VHL) is absent in the cancer cell or the cancer cell has a decreased expression or activity of Von Hippel-Lindau (VHL).

The cancer cell may be obtained from a subject, e.g., obtained from a cancer or tumor sample from a subject. The cancer cell may also be cancer cell line. The cancer cell may be a cell from a cancer sample or a cancer cell line which has been further engineered, e.g., to alter the expression levels of one or more genes (VHL, for instance).

The method may be an in vitro or in vivo method. An in vivo version of the method may comprise, for example, directly contacting an inhibitor of NF-κB signalling with a cancer cell in a subject. Alternatively, the inhibitor may be administered systemically to a subject with cancer. An in vitro version of the method may comprise, for example, contacting an inhibitor of NF-κB signalling with a cancer cell that has been cultured in vitro. The in vitro method may be a high-throughput method involving the screening of multiple NF-κB signalling inhibitors with one or more types of cancer cells.

Disclosed herein is a method of selecting a cancer subject for treatment with an inhibitor of NF-κB signalling, the method comprising a) detecting an indicator that is indicative of a level or activity of PB1 in a sample obtained from the subject, wherein the cancer is identified as being likely to be responsive to the inhibitor of NF-κB signalling when the level or activity of PB1 indicated by the indicator corresponds to an absence or decreased level or activity of PB1 in the sample as compared to a control; and b) selecting the cancer subject found likely to be responsive to treatment with the inhibitor of NF-κB signalling for treatment with the inhibitor of NF-κB signalling. In one embodiment, Von Hippel-Lindau (VHL) is absent in the cancer cell or the cancer cell has a decreased expression or activity of Von Hippel-Lindau (VHL).

Disclosed herein is a method of stratifying a cancer subject as a likely responder or non-responder to an inhibitor of NF-κB signalling, the method comprising detecting an indicator that is indicative of a level or activity of PB1 in a sample obtained from the subject, wherein the cancer is identified as being likely to be responsive to the inhibitor of NF-κB signalling when the level or activity of PB1 indicated by the indicator corresponds to an absence or decreased level or activity of PB1 in the sample as compared to a control, thereby stratifying the subject as a likely responder or non-responder to the inhibitor of NF-κB signalling. In one embodiment, Von Hippel-Lindau (VHL) is absent in the cancer cell or the cancer cell has a decreased expression or activity of Von Hippel-Lindau (VHL).

One or more additional criteria may be used along with PB1 level or activity for the selection and/or stratification of cancer subjects in the methods herein. Such criteria may include, for example, clinical parameters like histological findings or the levels of other biomarkers, and also the subject's treatment history with particular drugs or drug combinations.

The present disclosure also extends to kits for detecting an indicator that is indicative of a level or activity of Protein polybromo-1 (PB1) in a sample obtained from the subject. The kit may allow a cancer in a subject to be identified as being likely to be responsive to an inhibitor of NF-κB signalling when the level or activity of PB1 indicated by the indicator corresponds to an absence or decreased level or activity of PB1 in the sample as compared to the a control.

The kit may comprise one or more reagents or materials for detecting the indicator of a level or activity of PB1 and/or VHL in the sample obtained from the subject. For example, the kit may comprise reagents or materials for extraction of nucleic acid material (DNA or RNA) from a sample; nucleic acid primers for synthesizing a corresponding cDNA from a target RNA molecule; and nucleic acid labelled probes or primers for hybridizing with or amplifying the PBRM1 and/or VHL gene, transcript or cDNA. Alternatively or in addition, the kit may comprise labelled antibodies for detecting the PB1 and/or VHL protein.

The kits may also include appropriate reagents for detection of labels, positive and negative controls, washing solutions, blotting membranes, microtiter plates, dilution buffers and the like. For example, a nucleic acid-based detection kit may include (i) a polynucleotide encoding native PB1 (which may be used as a positive control), and (ii) a primer or probe that specifically hybridizes to a PB1 polynucleotide. Also included may be enzymes suitable for amplifying nucleic acids, including various polymerases (reverse transcriptase, DNA polymerase, DNA ligase etc. depending on the nucleic acid amplification technique employed), deoxynucleotides and buffers to provide the necessary reaction mixture for amplification. Such kits also generally will comprise, in suitable means, distinct containers for each individual reagent and enzyme as well as for each primer or probe. Alternatively, a protein-based detection kit may include (i) at least one native PB1 polypeptide (which may be used as a positive control), and (ii) one or more antigen-binding molecules that bind specifically to a PB1 polypeptide. The antigen-binding molecules are suitably detectably labeled. The kit can also feature various devices (e.g., one or more) and reagents (e.g., one or more) for performing one of the assays described herein, and/or printed instructional material for using the kit to quantify the level of PB1. The reagents described herein, which may be optionally associated with detectable labels, can be presented in the format of a microfluidics card, a chip or chamber, a microarray, or a kit adapted for use with RT-PCR, qPCR, qRT-PCR and similar techniques. The kit may also comprise instructions for selecting an appropriate inhibitor of NF-κB signaling based on the level or activity of PB1 indicated by the indicator.

Also provided herein is a composition comprising a) a sample obtained from a subject having a cancer that is likely to be responsive to treatment with an inhibitor of NF-κB signaling and b) a nucleic acid probe or primers for detecting or amplifying the PB1 gene or transcript, or antibodies for detecting the PB1 protein. The sample may be a cancer sample.

As used herein, “and/or” refers to and encompasses any and all possible combinations of one or more of the associated listed items, as well as the lack of combinations when interpreted in the alternative (or).

As used in this application, the singular form “a,” “an,” and “the” include plural references unless the context clearly dictates otherwise. For example, the term “an agent” includes a plurality of agents, including mixtures thereof.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

Those skilled in the art will appreciate that the invention described herein is susceptible to variations and modifications other than those specifically described. It is to be understood that the invention includes all such variations and modifications which fall within the spirit and scope. The invention also includes all of the steps, features, compositions and compounds referred to or indicated in this specification, individually or collectively, and any and all combinations of any two or more of said steps or features.

Unless otherwise defined, all technical and scientific terms used herein have the same meanings as commonly understood by one of ordinary skill in the art to which this invention belongs.

Certain embodiments of the invention will now be described with reference to the following examples which are intended for the purpose of illustration only and are not intended to limit the scope of the generality hereinbefore described.

### EXAMPLES

### Materials

### Cell Lines

HK2 (CRL-2190), Primary renal proximal tubule epithelial cells (PCS-400-010), 786-O (CRL-1932), 769-P (CRL-1933) were purchased from ATCC and maintained with RPMI (Gibco)+10% FBS at 37 °C. NCC432 and NCC2112 were derived from patient tumors. Briefly, tumor cells were dissociated from primary tumors by collagenase, seeded and maintained in RPMI with 10% FBS. At 80-90% confluency, the cells were passaged at 1:3 ratio. The cultured cells were considered to be successfully immortalized after 60 passages.

### CRISPR-Cas9 Mediated Gene Editing

PBRM1 was knocked out using CRISPR-Cas9 mediated gene editing in 786-0, 769-P and HK-2. For 786-O and HK-2 cells, phosphorylated and annealed sense and antisense oligos were ligated into Bpil digested vectors. Left gRNAs were cloned into the Bpil digested pX330A-2A GFP-1X2 backbone (Addgene #58766, with addition of GFP by Dr. Shang Li, DUKE-NUS, Singapore) whereas the right gRNAs into Bpil digested pX330S backbone (Addgene #58778). Golden gate assembly was performed to assemble the 2 gRNA protospacers into the pX330A-2A-GFP-1X2 plasmid backbone using a one-step digestion and ligation with slight modifications. After transfection using Lipofectamine 3000 (Life Technologies), GFP-positive single cells were sorted and cultured. Individual clones were validated for enhancer deletion by PCR of genomic DNA and the resulting protein expression was measured using immunoblotting. Three individual knockout clones were pooled to form the final knockout cell line. Late passage (passage>30) PBRM1 KO 786-O cells were used for functional studies.

The gRNAs used for deletion of PBRM1 are:

Left (5′-3′, hg19 chr3: 52, 682, 407-52,
682, 424):
(SEQ ID NO: 3)
CACCGCGAGGAGATCTATATCTT
Right (5′-3′, hg19 chr3: 52, 683, 098-52,
683, 117):
(SEQ ID NO: 4)
CACCGACCTTCCATTTCGCTCAGTT

For CRISPR mediated deletion in 769-P cells, pUC_AIO_CAS9_EF1T2GFP_tandem_gRNA was used to clone multiple pairs of gRNAs against PBRM1 or ARID2. The sequences are as follows:

| Gene | Clone # | First gRNA | Second gRNA |
|---|---|---|---|
| PBRM1 | clone1 | TGGCTGCAGCCAAGTTGGA | ATAACACCTAACCTAACAT |
| | | TGG (SEQ ID NO: 5) | GGG (SEQ ID NO: 6) |
| | clone2 | ATAGGCATTTGATAGTACT | GTTACCAATACAATTGCAC |
| | | TGG (SEQ ID NO: 7) | AGG (SEQ ID NO: 8) |
| | clone3 | ATTGACATGGGTTTAGCGT | AAGGGATCCTAGGATATCC |
| | | TGG (SEQ ID NO: 9) | TGG (SEQ ID NO: 10) |
| | clone4 | TGGCTGCAGCCAAGTTGGA | CCTCTCTAAGATGTAAACT |
| | | TGG (SEQ ID NO: 11) | AGG (SEQ ID NO: 12) |
| ARID2 | clone1 | ATCTATCAAGCTACCTGCT | AGGGTCCAATCAGGCTATG |
| | | AGG (SEQ ID NO: 13) | AGG (SEQ ID NO: 14) |
| | clone2 | ATCTATCAAGCTACCTGCT | AGGGTCCAATCAGGCTATG |
| | | AGG (SEQ ID NO: 15) | AGG (SEQ ID NO: 16) |
| | clone3 | CTGTATAAACCGAGGGCGA | AGGGTCCAATCAGGCTATG |
| | | CGG (SEQ ID NO: 17) | AGG (SEQ ID NO: 18) |

Stable and inducible knockdown of ARID2 and SMARCA4 2 in cell lines Stable knockdown of ARID2 and SMARCA4 was performed using retroviral transduction with predesigned MISSION shRNA constructs (Sigma Aldrich) with the following sequences:

shARID2-1(TRCN0000162164):
(SEQ ID NO: 19)
CCGGCCAGCGTGAAATGTATCCATTCTCGAGAATGGATACATTTCACGCTG
GTTTTT
shARID2-2(TRCN0000166264):
(SEQ ID NO: 20)
CCGGCGTACCTGTCTTCGTTTCCTACTCGAGTAGGAAACGAAGACAGGTAC
GTTTTT
shARID2-3(TRCN0000166359):
(SEQ ID NO: 21)
CCGGCCGACTAACAGCTGCCTTAATCTCGAGATTAAGGCAGCTGTTAGTCG
GTTTTT
shSMARCA4-2(TRCN0000218544):
(SEQ ID NO: 22)
CCGGTCAAACAGTACCAGATCAAAGCTCGAGCTTTGATCTGGTACTGTTTG
ATTTTT
shSMARCA4-3(TRCN0000231101):
(SEQ ID NO: 23)
CCGGCTTTGCGTATCGCGGCTTTAACTCGAGTTAAAGCCGCGATACGCAAA
GTTTTT

MISSION shRNA glycerol stock was cultured, and its plasmid were extracted using NucleoBond® Xtra Maxi Plus EF (MACHEREY-NAGEL). Lentiviruses were packaged with packaging plasmid psPAX2 (Addgene #12260) and the envelope plasmid pMD2.G (Addgene #12259), and transfected into HEK293T using Lipofectamine 3000 (Life Technologies). The supernatant consisting of lentiviral particles was harvested and filtered 48 hours posttransfection. It was inoculated onto cell lines, in which they were then selected with puromycin (2 μg/mL for 786-O or 10 μg/mL for NCC432) for 3 days. Cells were analyzed for gene and protein expression, and other functional assays post selection. Off-target toxicity effects were assessed on ARID2-null cell line (NUGC3) and SMARCA4-null cell line (A549).

Dox-inducible shRNA constructs were made by ligating annealed sense and antisense oligos into Agel and EcoRI digested Tet-pLKO-puro plasmid (Addgene #21915). Packaging of the viral particles and transduction of cells was as described above. Additionally, these knockdown cells required doxycycline treatment (1 μg/mL) for 2 days to induce shRNA expression before subsequent analysis.

### Re-Expression of PBI

Wildtype PBRM1 cDNA was reversed transcribed from the RNA extracts of normal kidney tissue, and cloned into either pBABE vector or pLenti-GFP (Addgene 17448). Retroviruses were packaged using Platinum-A (Plat-A) Retroviral Packaging Cell Line (Cell Biolabs). Lentiviruses were packaged with packaging plasmid psPAX2 (Addgene 12260) and the envelope plasmid pMD2.G (Addgene 12259). Cells were selected with puromycin for 3 days. For rescue experiments in 786-O KO cells, RNA of PB1-expressing cells was harvested at very early passage (passage 1-2).

### Drug Treatment

In 6 wells, 1,000 786-O cells or 2,500 NCC432 cells were seeded and treated with DMSO control or increasing concentration of drugs for 7 days and stained with crystal violet. For drug screening, we used SGC epigenetic probe collection. For RNA-seq with bortezomib treatment, 2×10⁵ 786-O cells were treated with DMSO or 0.005 μM bortezomib and harvested 9 hours after treatment for RNA extraction. For RT-qPCR with SMARCA2/4 ATPase inhibitor treatment, 1×10⁵ 786-O cells were treated with DMSO or 0.01 μM SMARCA2/4 ATPase inhibitor (Targetmol, T10616, CAS: 2270879-17-7) for 3 days before RNA extraction.

### In Vivo Tumor Formation

All animal studies were conducted in compliance with animal protocols approved by Institutional Animal Care and Use Committee (IACUC) of Singapore. Female NOD/SCID mice (8-10 week old) were implanted subcutaneously with 1×10⁶ 786-0 cells with either wildtype PBRM1 or PBRM1 knocked out subcutaneously. Male NSG mice (The Jackson laboratory) were implanted with 1×10⁶ A498, NCC2112 or NCC432 with or without PBRM1. Tumor volume was monitored every 3-4 days. Tumor volume was calculated as (length×width×width)×π/6. Animals were sacrificed when the tumor volume exceeded 2000 mm³.

### In Vivo Drug Treatment

Bortezomib (0.05 mg/kg) was given twice weekly (day1, day 4) to male NSG mice (The Jackson laboratory) via the tail vein. Tumor volume and body weight were monitored every 3-4 days.

### ChIP-Seq

For each protein of interest, approximately 2×10⁷ cells were cross-linked with 1% formaldehyde for 10 min at room temperature, and stopped by adding glycine to a final concentration of 0.2M. Chromatin was extracted and sonicated to ~500 bp (Vibra cell, SONICS). The following antibodies were used for chromatin immunoprecipitation: PBRM1 (Bethyl A301-591A Lot: 3-4), SMARCA4 (AbCAM ab110641 Lot: GR3208604-8), ARID2 (Bethyl A302-230A Lot: 2), BRD7 (Bethyl A302-304A Lot: 1), ARID1A (Santa Cruz sc-32761 Lot: #H2217), SMARCC1 (Santa Cruz sc-32763 Lot: #K1617), NFkB p65/RELA (Santa Cruz sc-372 Lot: #J1915; Diagenode C15310256, Lot: 24468; Invitrogen 51-0500, Lot: WG331314), BRD9 (Bethyl A303-781A, Lot2), RNA POL II (Merck Millipore 05-623 Lot: 2958013), H3K27ac (AbCAM ab4729-100 Lot: 741806), H3K14ac (AbCAM ab52946 Lot: GR3252548-5), H3K4me3 (Merck Millipore 07-473 Lot: 2930138), H3K4me1 (AbCAM ab8895 Lot: GR312093-1), H3K27me3 (Millipore 07-449 Lot: JBC1924326), H3K9me3 (Millipore 07-442 Lot: DAM1411287). The total volume of immunoprecipitation was 2 ml and the amount of antibody used was 20 μg. The input DNA was precleared with protein G Dynabeads (LifeTechnologies) for 2 hours at 4 °C and then incubated with antibodies overnight at 4 °C. Protein G beads were added the following day and mixture was nutated for 3 hours at 4 °C. The beads were washed 6 times with wash buffers at 4 °C temperature. At least 10 ng of the amplified DNA was used with NEBNext ChIP-Seq library prep reagent set (NEB). Each library was sequenced to an average depth of 30-50 million reads on HiSeq2000, HiSeq4000 or Novaseq 6000.

### ChIP-Seq Analysis

Sequencing tags were mapped against the human reference genome (hg19) using Burrows-Wheeler Aligner (BWA-mem) (version 0.7.10). Only reads with mapQ>10 and with duplicates removed by rmdup were used in the subsequent analysis. Significant peaks were called using MAC1037 (version 2.0.10.20120913, q-value<0.01). Peaks present in both PB1 wildtype and knockout cells were treated as non-specific regions and removed. ChIP-seq profiles were plotted using NGSplot (version 2.47) and deeptools (version 3.3.0). Differential ChIP-seq peaks were called using DiffBind (version 3.0) after performing background normalization. Distribution of PBRM1 ChIP-seq binding sites was annotated using ChIPseeker (version 1.22.1). Motif analysis was performed with HOMER (version 4.7.2).

### Differential Peak Pathway Analysis

Differential peaks were assigned to the nearest genes. Pathway enrichment based on assigned genes was performed by ChIP-enrich.

### RNA-Seq

Total RNA was extracted using the Qiagen RNeasy Mini kit. RNA-seq libraries were prepared using Illumina Tru-Seq Stranded Total RNA kit protocol, according to the manufacturer's instructions (Illumina, San Diego, California, USA). The libraries were created with 1 μg of total RNA. Libraries were validated with an Agilent Bioanalyzer (Agilent Technologies), diluted and applied to an Illumina flow cell using the Illumina Cluster Station. Sequencing was performed on an Illumina HiSeq4000 sequencer with the paired-end 150 bp read option.

RNA-Seq Pathway Analysis RNA-seq data were mapped to hg19 (h117d5) using STAR, with GENCODE annotation (gencode.v19.annotation.gtf) and recommended settings from STAR manual. Gene expression levels were quantified with STAR. Log fold change of differential expression genes was computed with DESeq2. Gene Set Enrichment Analysis (GSEA) was performed using GSEA (v4.0.3). Heatmaps were generated using pheatmap (version 1.0.12).

### ATAC-Seq

ATAC-seq was performed based on previous protocol published by Buenrostro et al. Briefly, cells were trypsinized and digested with Tn5 transposase (Illumina) for 30 mins. Fragments were pre-amplified with NEBNext High-Fidelity 2×PCR Master Mix (NEB). Optimal PCR amplification cycle was pre-determined based on 1/4 of the maximal intensity of a RT-PCR reaction.

### ATAC-Seq Analysis

Analysis was adapted from previous protocol published by Buenrostro et al. Adapters were removed by Cutadapt. Pair-end reads were mapped to hg19 using Bowtie using the parameter −x2000. Only reads mapQ>30 were used, and duplicates were removed by rmdup. Reads mapped to ChrM and ChrY were removed. Library size was estimated using PICARD tools (http://broadinstitute.github.io/picard/). Peaks were called using MAC10 (q<0.01). Nucleosome positioning was determined using NucleoATAC.

### ChIP-Mass Spectrometry

We first performed ChIP according to the protocol above. Subsequently, we washed protein bound beads with PBS 3 times. Proteins were de-crosslinked with 50 mM Tris buffer PH 8.0 at 70 °C for 3 hours. Proteins were reduced by incubating with 20 mM TCEP at 55 °C for 20 min, and then alkylated with 55 mM CAA for 30 min at room temperature in the dark. Each reaction was topped up to a volume of 600 μl with 100 mM TEAB, pH 8.5 buffer. Each sample was digested with 10 μg trypsin (Promega) overnight at 37 °C. Supernatant was acidified with 1% TFA and desalted on HLB C18 columns (Waters). The column was first activated with 500 μl acetonitrile and equilibrated with 500 μl 0.5% acetic acid. After loading the sample to the column, the column was washed once with 0.5% acetic acid and digested peptides were eluted using 0.5% acetic acid in 80% acetonitrile. Digested peptides were separated on an Easy-Spray reversed-phase analytical column (50 cm×75 μm internal diameter, C18, 2 μm particles, Thermo) using a 70 min gradient (0.1% formic acid in water and 0.1% formic acid in 99% acetonitrile as mobile phases) on EASY-nLC 1000 liquid chromatography system. Data was acquired using an Orbitrap Fusion™ Tribrid™ Mass Spectrometer (Thermo Scientific) with the following settings: source spray 2.1 kV, RF=55, survey scan MS1 in Orbitrap analyzer resolution=60 k with AGC target of 400 k ions, MS2 in iontrap analyzer (scan rate=Normal, AGC target 10 k). Data was acquired in DDA speed mode with 3 seconds cycle. Proteins were identified and LFQ intensities were used for quantitation using MaxQuant version 1.6.5.0 with the following parameters: Fix modifications (Carbamidomethyl (C), Variable modifications Oxidation (M), Deamidation (NQ), Acetylation (N-Term Protein) Mass accuracy MS: 4.5 ppm (after recalibration), MS/MS: 20 ppm, missed cleavages (2), FDR for peptide and protein level 1%. Data was searched against Human Uniprot database. Raw data is deposited at JPOST repository (see Data Accession).

### Co-Immunoprecipitation

Nuclear proteins were isolated using NE-PER Nuclear and Cytoplasmic Extraction Reagents (Pierce) and diluted to 1 mg/mL using nuclear extraction buffer. A total of 5 μg antibodies were added to 250 μL of nuclear protein and incubated overnight. Beads were washed 3 times with RIPA buffer. SDS loading buffer was added to beads and heated for 10 mins at 95 °C.

### Immunoblotting

Cell lines were harvested with cold RIPA lysis buffer (50 mM Tris pH 8, 150 mM NaCl, 0.1% Triton X-100, 0.5% Sodium deoxycholate, 0.1% SDS) with protease inhibitors (Roche) on ice. Cells were mechanically lysed by passing through a 25 Gauge needle and centrifuged at 13,000 rpm for 15 min at 4 °C. Protein concentration was measured by Pierce BCA protein assay (Life Technologies). Cell lysates were heated at 70 °C for 10 min in sample buffer. Per well, 15 μg of cell lysate was loaded and gel electrophoresis was run at 130V constant for 90 mins. Proteins were transferred to nitrocellulose membranes by transferring at 100V for 100 mins in ice. Western blotting was performed by incubating membranes overnight at 4 °C with the following antibodies and dilutions: PBRM1 (Bethyl A301-591A, Lot: 3-4, 1:1000; Cell Signaling 91894S Lot: 1), SMARCA4 (Santa Cruz sc-17796 Lot: #K2917, 1:1000; AbCAM ab110641 Lot: GR3208604-8), ARID2 (Santa Cruz sc-166117 Lot: #GR2712, 1:1000; Bethyl A302-230A Lot: 2), BRD7 (Cell Signaling 14910S Lot: 1; Bethyl A302-304A Lot: 1), ARID1A (Santa Cruz sc-32761 Lot #H2217, 1:1000), ARID1B (AbCAM ab59461 Lot: GR3216851-1, 1:1000) and SMARCC1 (Santa Cruz, sc-32763 Lot: #K1617; Bethyl A301-019A-M Lot: 1), ACTL6A (Santa Cruz sc-47808 Lot: #F1511, 1:1000; Bethyl A301-391A-M Lot: 2; Santa Cruz sc-137063 Lot: #D0109), SMARCE1 (AbCAM ab131328 Lot: GR97519-10, 1:1000) and SMARCB1 (AbCAM ab126734 Lot: GR95565-9, 1:1000; Santa Cruz sc-166165 Lot: #G1916), NFkB p65 (Santa Cruz sc-372 Lot: #J1915), RNA POL II (Merck Millipore 05-623 Lot: 2958013), H3K27ac (AbCAM ab4729-100 Lot: 741806), H3K14ac (AbCAM ab52946 Lot: GR3252548-5), H3K4me3 (Merck Millipore 07-473 Lot: 2930138), H3K4mel (AbCAM ab8895 Lot: GR312093-1), H3K27me3 (Millipore 07-449 Lot: JBC1924326), H3K9me3 (Millipore 07-442 Lot: DAM1411287), VHL (Cell Signaling #2738 Lot: 1), Phospho-NFkB (Cell Signaling #3031 Lot: 10), RelB (Bethyl A302-182A Lot: 2), NFkB1 (Santa Cruz sc-8414 Lot: #H2420), NkKB2 (Santa Cruz sc-7386 Lot: #F3020), Anti-α Tubulin Antibody (Santa Cruz sc-8035 Lot: #E1713), Anti-Lamin A+Lamin C antibody (AbCAM ab108595), Histone H3 (Cell Signaling #4499), Anti-β-Actin antibody (Santa Cruz sc-47778), Normal Rabbit IgG (Merck Millipore 12-370 Lot: 2843037), Normal Mouse IgG (Merck Millipore 12-371 Lot: 3045579), Anti-Rabbit IgG (H+L) (Promega W4011), Anti-Mouse IgG (H+L) (Promega W4021). Membranes were incubated in secondary antibodies at 1:10,000 dilution for 1 hr at room temperature and developed with SuperSignal West Femto Maximum Sensitivity Substrate (Thermo Scientific).

### Example 1

### PB1 is Tumor Suppressive

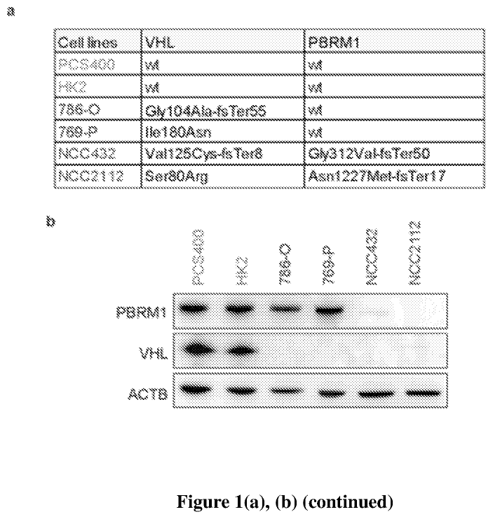

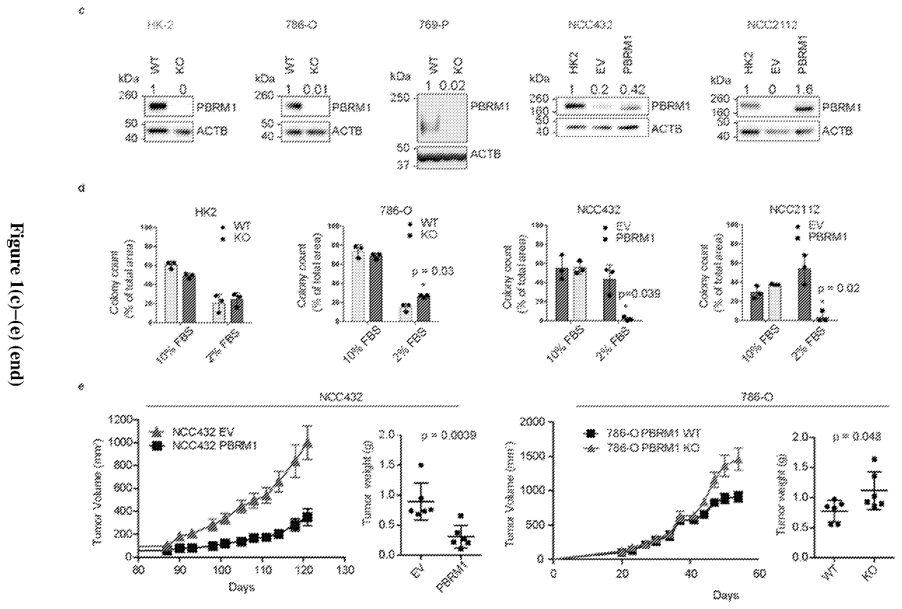

A panel of cell lines was assembled representing 1) normal kidney epithelial cell lines proficient in both VHL and PB1 (PC1200, HK2), 2) ccRCC cell lines deficient in VHL but proficient in PB1 (786-O and 769-P) and 3) ccRCC cell lines deficient in both VHL and PB1 (NCC2112 and NCC432) (FIG. 1A). NCC010 and NCC432 were derived in-house from patient tumors and harbor truncating mutations in PB1 with >97% alternative allele frequency (FIG. 1A). At the protein level, NCC2112 exhibit complete loss of PB1 while NCC432 retains residual amount of PB1 protein (FIG. 1B).

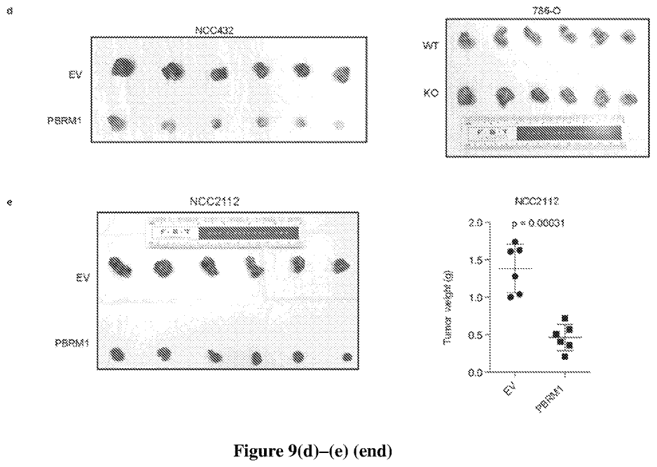

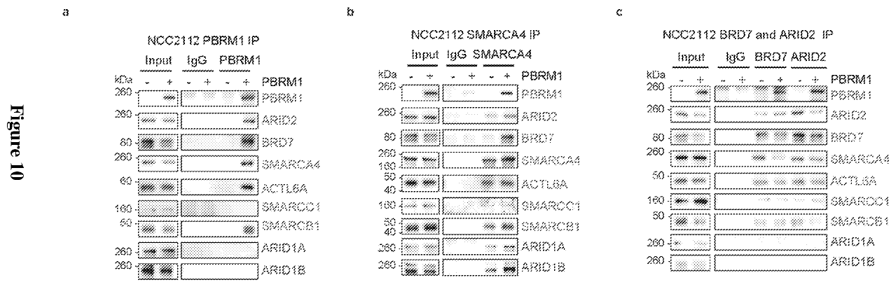

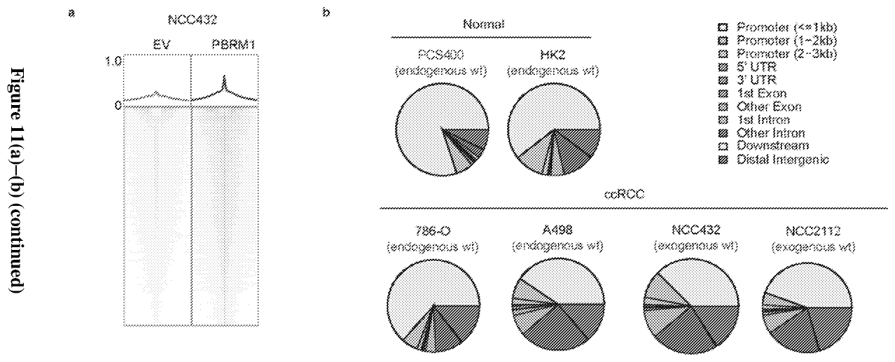

CRISPR-Cas9-mediated gene editing was utilized to knock out PB1 in PB1-proficient cell lines and to reciprocally re-express physiological levels of PB1 in PB1-deficient cell lines (FIG. 1C, 9A). Stable loss of PB1 did not promote colony formation under normal in vitro conditions but enhanced clonogenic potential of ccRCC cell lines under low serum conditions (FIG. 1D, 9B). PB1 restoration also suppressed soft agar colony formation of PB1-deficient cells (FIG. 9C). Lastly, tumor formation studies showed that PB1 restoration led to significant delays in tumor formation for both NCC432 and NCC2112, as measured by tumor volume and tumor weight (FIG. 1E, 9D, 9E). Conversely, the loss of PB1 in 786-O increased tumor growth (FIG. 1E, 9D), which is consistent with previous results. These data demonstrated unequivocally the tumor repressive nature of PB1.

### PB1-Deficiency Disengages BRD7 but Retains ARID2 and SMARCA4

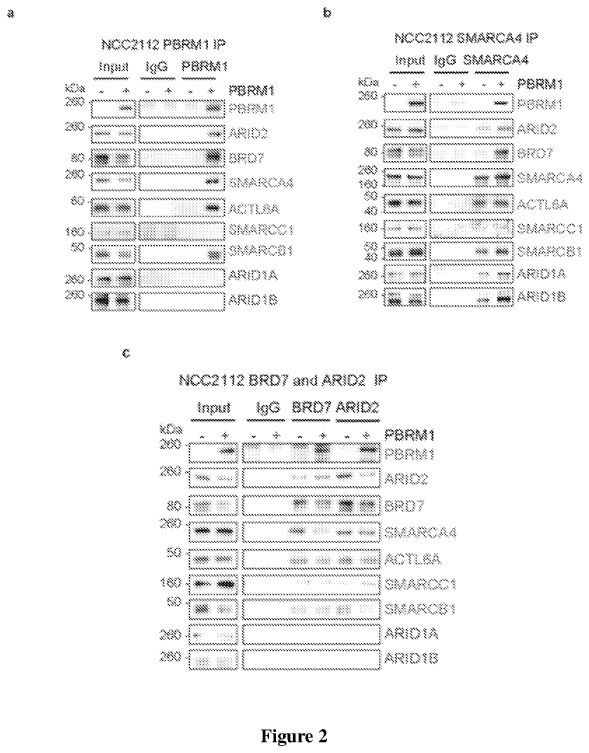

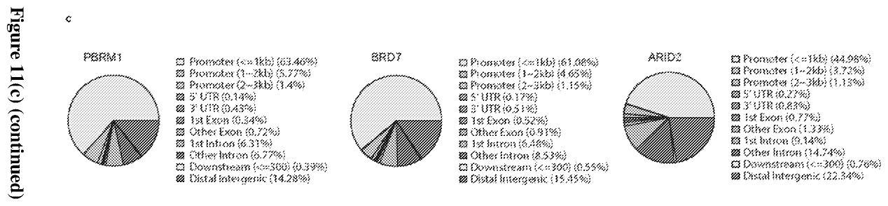

The molecular composition of PBAF complexes was examined after PB1 loss. Immunoprecipitation of PB1 in soluble nuclear lysates pulled down PBAF-specific accessory subunits (ARID2 and BRD7), ATPase subunit (SMARCA4) and core SWI/SNF subunits (SMARCC1, SMARCB1 and ACTL6A), but not BAF-specific accessory subunits (ARID1A/B) (FIG. 2A). Label-free ChIP mass-spectrometry was performed to identify interaction partners of PB1 on the chromatin. After filtering for specificity (WT/KO>10), the top 9 out of the 10 most abundant interaction partners were subunits of the SWI/SNF complex, especially of the PBAF subclass (FIG. 2B). Similarly, PB1 re-expression but not empty vector control pulled down PBAF-specific subunits in NCC2112 cells (FIG. 10A).

Co-immunoprecipitation and ChIP mass spectrometry of SMARCA4 showed that PB1 loss severely disrupted the association between BRD7 and SMARCA4 but preserved the association between SMARCA4 and ARID2 and other SWI/SNF subunits (FIG. 2C, 2D). The integrity of the PBAF complex was further examined by performing co-immunoprecipitation of BRD7 and ARID2. Co-immunoprecipitation of BRD7 pulled down reduced amount of ARID2 and reciprocally, co-immunoprecipitation of ARID2 pulled down reduced amount of BRD7 in PB1-deficient 786-O (FIG. 2E). The trend was reproducible in NCC2112 (FIG. 10B, C). The loss of PB1 also resulted in significantly reduced association between the core subunits (SMARCC1, SMARCB1 and ACTL6A) and BRD7 and ARID2 in 786-0. The above results implied that PB1-deficient PBAF complex experienced compromised integrity, disengagement of BRD7 from SMARCA4 but preserved association between ARID2 and SMARCA4.

### PB1 Binds to Promoter-Proxy Regions

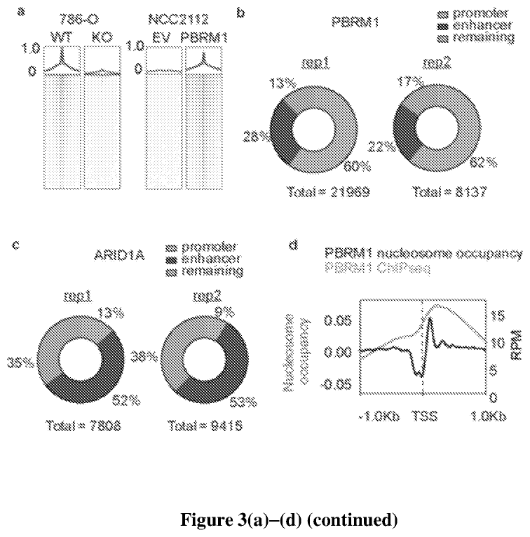

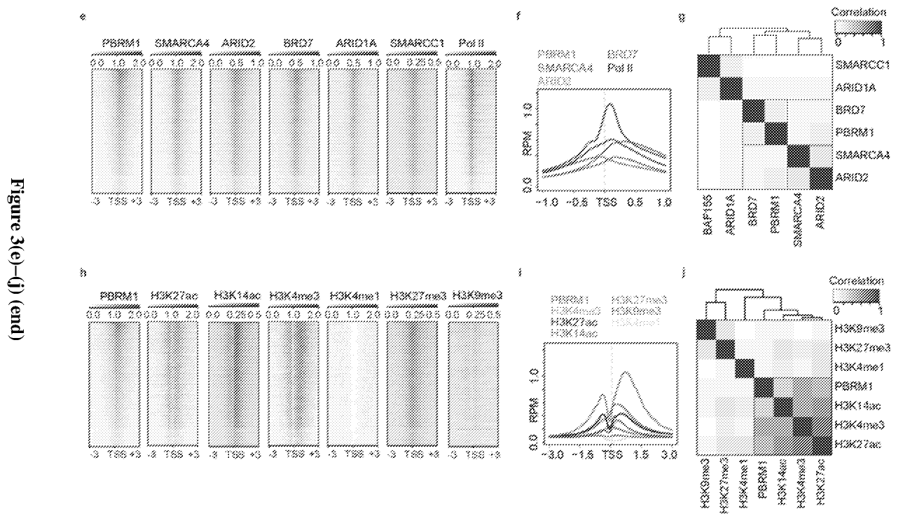

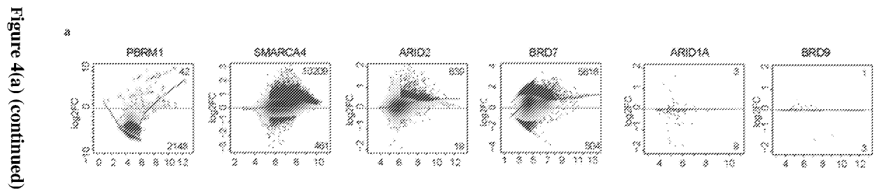

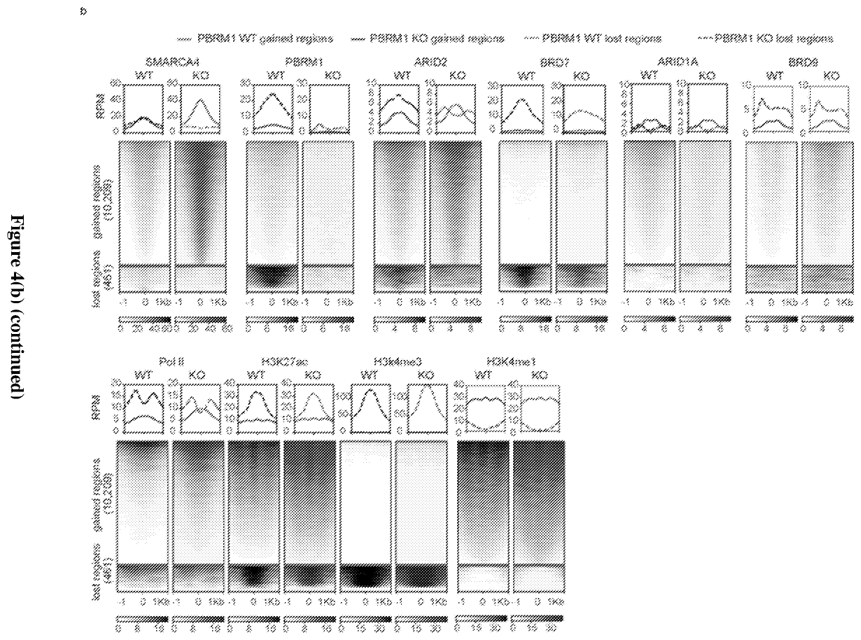

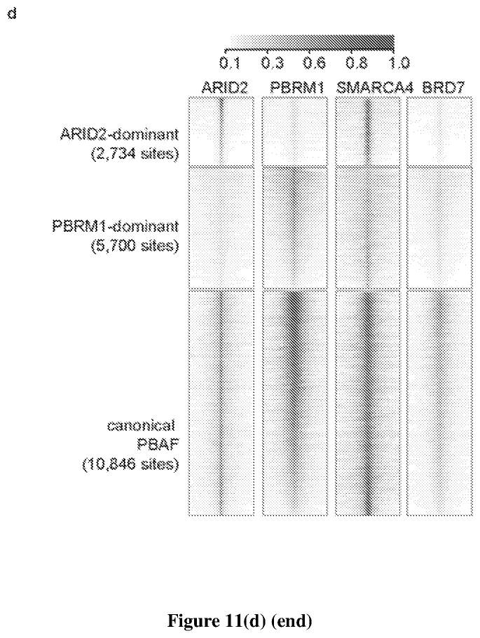

Since the genomic localization of endogenous PB1 in kidney cells has not been determined previously, a comprehensive survey of PB1 chromatin profiles was conducted across all 6 cell lines. The ChIP-seq signals were highly specific (FIG. 3A, FIG. 11A). Genomic distribution of ChIP-seq peaks indicated a preponderance of PB1 enrichment at promoter regions (FIG. 3B). In contrast, ARID1A, the accessory subunit of BAF complexes, occupied more frequently at intronic and distal intergenic regions (FIG. 3C). A closer examination revealed that PB1 occupied downstream of transcription start sites (TSS) (FIG. 3D); superimposing nucleosome positioning with PBRM1 ChIP-seq placed PB1 binding between +1 to +2 nucleosomes (FIG. 3D). The occupancy of PB1 showed that, distinct from the enhancer-centric BAF complexes, PBAF-specific SWI/SNF complexes predominantly occupy promoter regions.

ChIP-seq of other SWI/SNF subunits revealed an offset between PB1 and ARID2, with ARID2 located upstream of TSS and PB1 and BRD7 downstream of RNA polymerase II (FIG. 3E, 3F). Hierarchical clustering also showed a greater similarity between PB1 and BRD7 occupancy compared to ARID2, with PB1 and BRD7 mainly occupying promoters and ARID2 occupying both promoters and distal regions (FIG. 3G, FIG. 11C). Even though the majority of the genomic regions were bound by all PBAF subunits (10,846 sites), a significant number of sites were detected which were occupied by PBAF subcomplexes with 2,734 ARID2high/PBRMllow sites and another 5,700 PB 1high/ARID2low sites (FIG. 11D). Taken together, our ChIP-seq profiles suggest that PBAF complexes consist of a submodule of PB1 and BRD7, separate from ARID2.

In terms of histone modifications, PB1 binding sites were enriched with promoter mark (H3K4me3) but were depleted of enhancer mark (H3K4me1), consistent with its promoter-centric occupancy (FIG. 3H). PB1 also colocalized with H3K14ac (FIG. 3I), which is known to associate with PB1 via its bromodomains 2, 4 and 5 with high affinity. PB1 was depleted of the heterochromatin mark H3K9me3 but was slightly enriched for the repressive mark H3K27me3. Hierarchical clustering confirmed the close association between PB1 and active promoter marks (H3K4me3, H3K27ac and H3K14ac) and separation of PB1 from heterochromatin marks (H3K9me3 and H3K27me3) and enhancer marker (H3K4me1) (FIG. 3J). Therefore, PB1 localized to active promoter regions downstream of transcription start sites and was closely associated with BRD7.

### PB1-Deficient PBAF Complexes Target De Novo Distal Regions

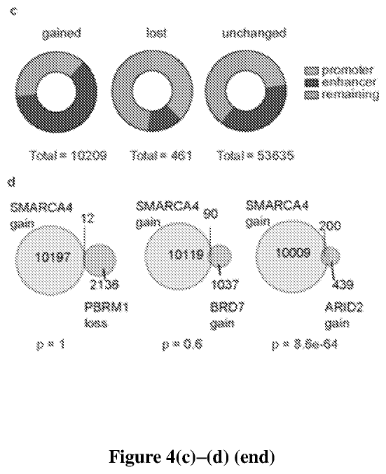

The way in which PB1 loss altered chromatin binding of PBAF subunits was examined next. The most drastic change was the gain of SMARCA4 binding, observed in ~1/6 of total SMARCA4 peaks (10,209 gained peaks, 461 lost peaks and 53,635 unchanged peaks, absolute fold change>1.5, q<0.1, negative binomial test) (FIG. 4A). ARID2 also gained genomic occupancy albeit to a less extent than SMARCA4 (639 gained, 19 lost and 20,379 unchanged). BRD7, despite being disengaged from SMARCA4, similarly gained occupancy (1,128 gained, 30 lost and 2,023 unchanged). In contrast, significant alterations in ARID1A and BRD9, subunits of the BAF and ncBAF complexes respectively, were not observed. This implies that PB1 loss primarily impacted the PBAF subunits.

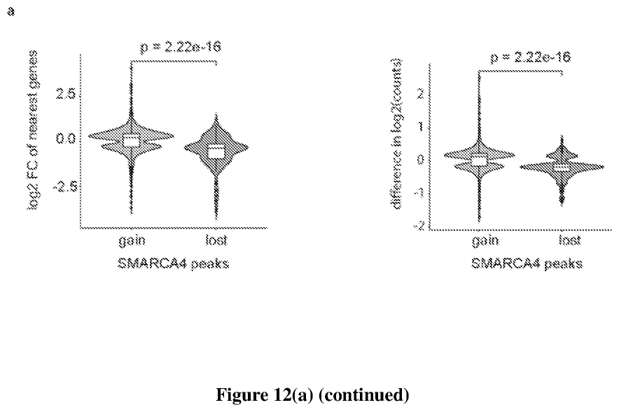

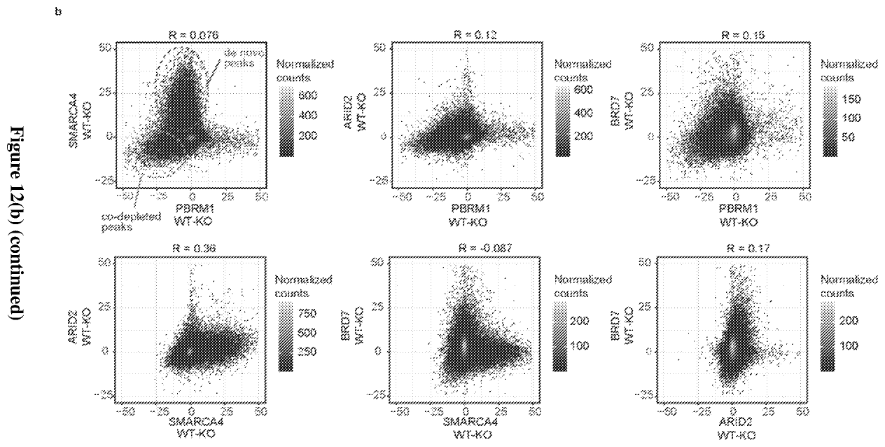

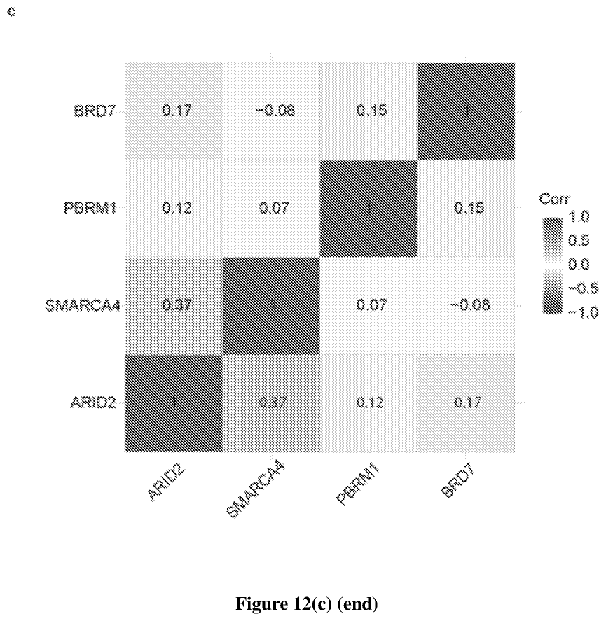

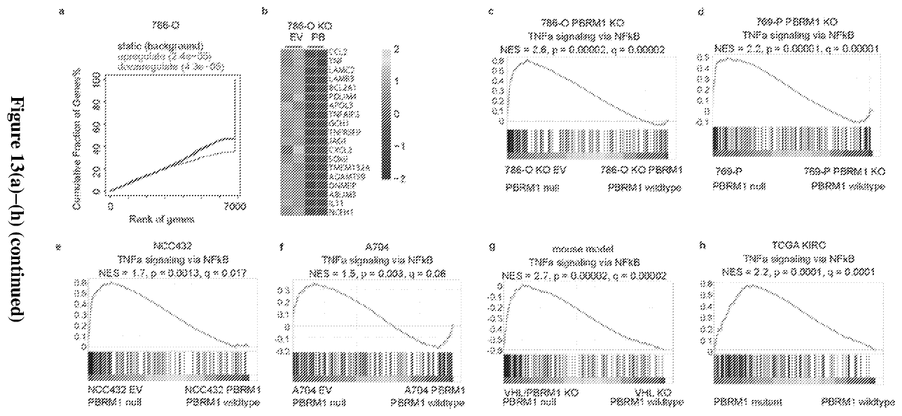

Focusing on SMARCA4, the newly gained regions were associated with increased ARID2 and Pol II occupancy (FIG. 4B). The lost SMARCA4 regions were accompanied by synchronous depletion of other PBAF subunits. The gained SMARCA4 peaks fell largely in enhancer regions enriched with H3K4mel (77.3% enhancers vs. 18.8% promoters) whereas the lost SMARCA4 peaks were found largely in promoter regions enriched with H3K4me3 (82.6% promoter vs. 6.0% enhancer, FIG. 4B, C). Compared to common regions, the distribution of promoters and enhancers were statistically skewed at both gained and lost SMARCA4 regions (p<0.0001, chi-square test). SMARCA4 appeared to activate gene expression because genes mapped to gained SMARCA4 regions gained expression compared to genes mapped to lost regions (p<2.2 e-16, two-sided Wilcoxon test) (FIG. 12A).

The gained SMARCA4 regions showed minimal overlap with lost PB1 (90/1127 peaks, p=1) or gained BRD7 peaks (90/1127 peaks, p=0.6), and instead overlapped significantly with gained ARID2 peaks (200/639 peaks, p=8.6e-64) (FIG. 4B, FIG. 4D). When changes in occupancy at regions bound by PBAF subunits was examined, both ARID2 and SMARCA4 gained maximal binding at regions corresponding to no change in PBRM1 (WT-KO=0) whereas PB1 and BRD7 were coordinated in the loss of occupancy (FIG. 12B). The above ChIP-seq results suggest that PB1 loss resulted in redistribution of SMARCA4 to de novo regions.

### PB1 Loss Activates the NF-κB Pathway

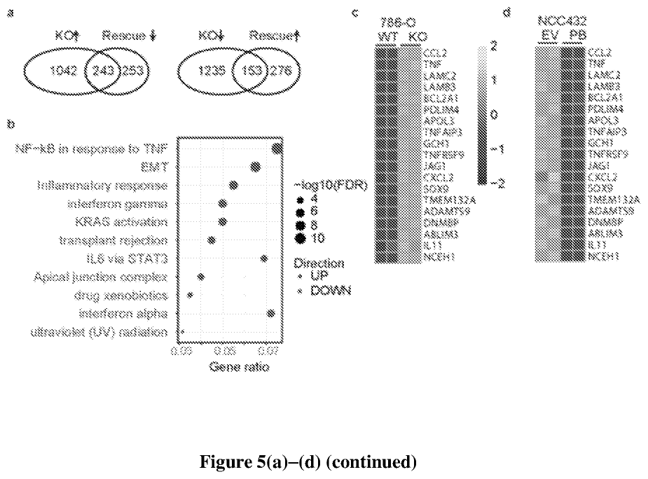

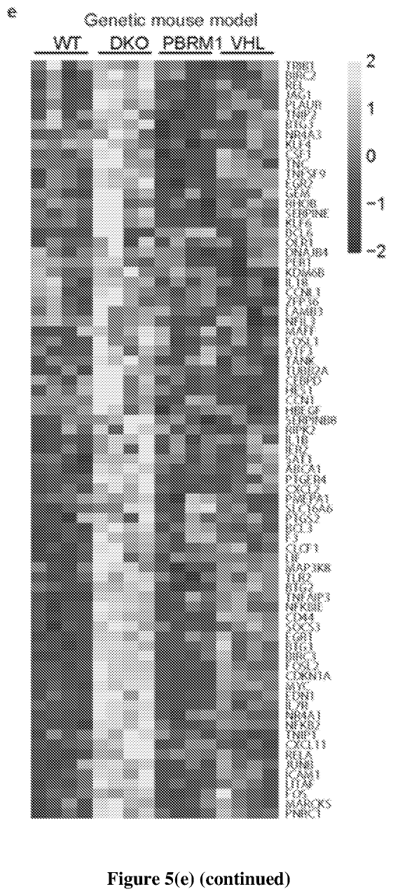

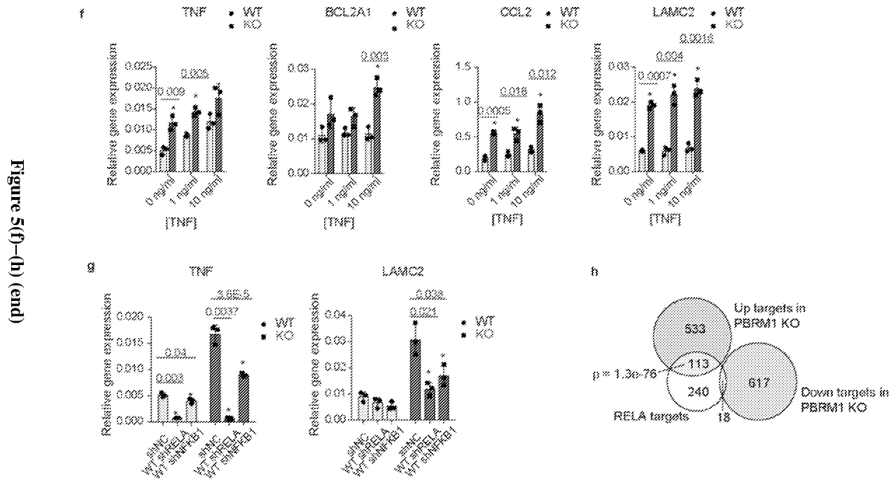

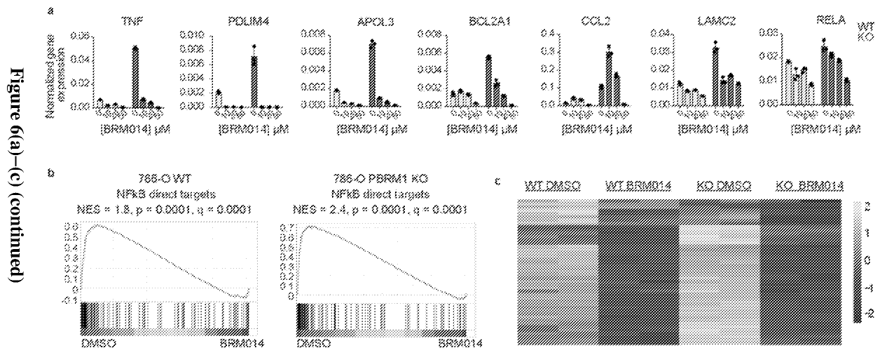

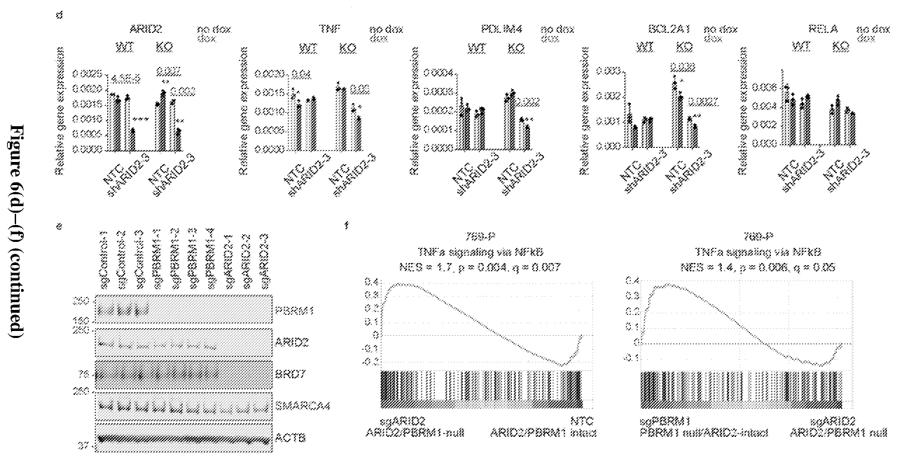

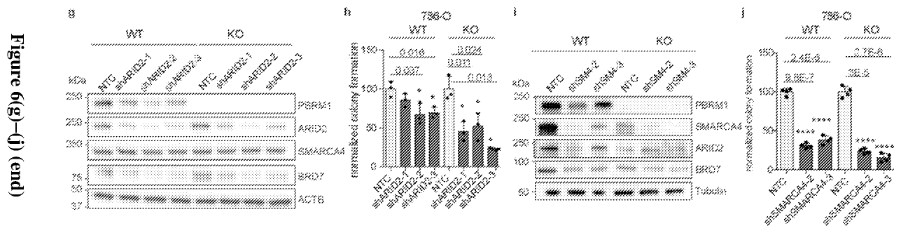

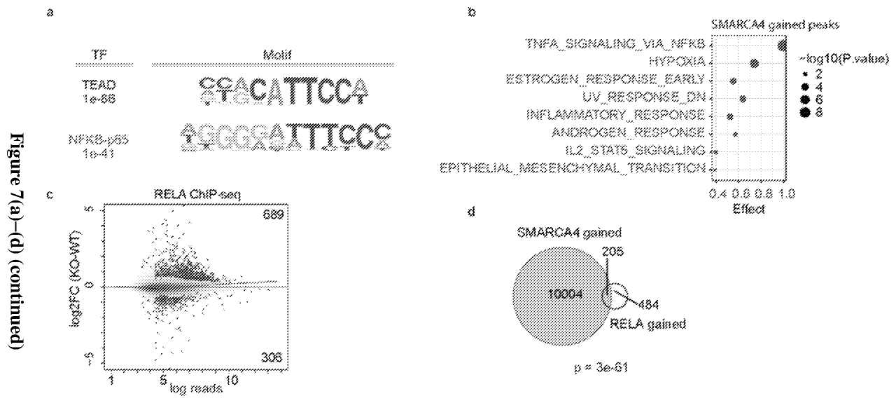

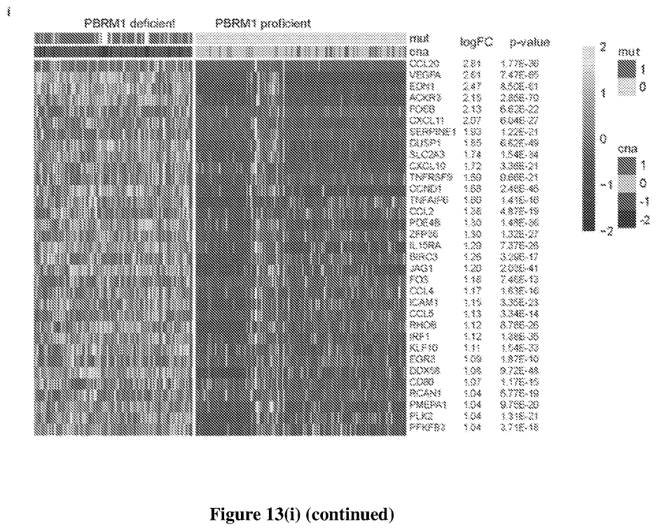

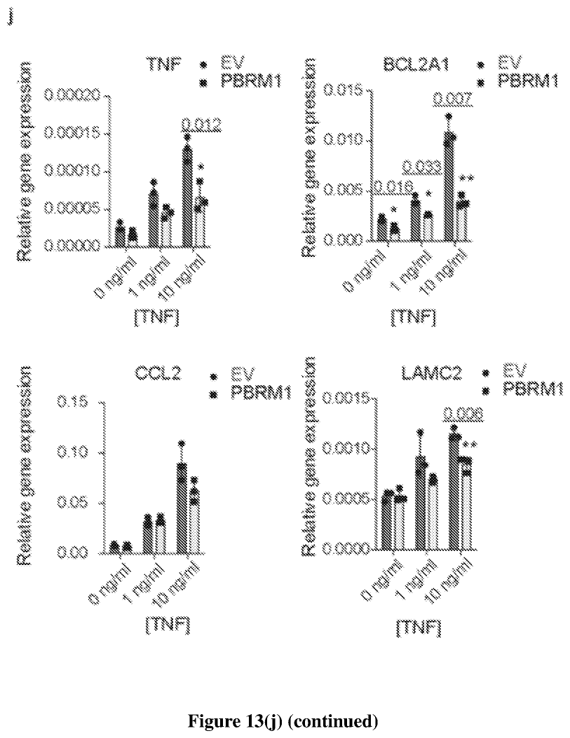

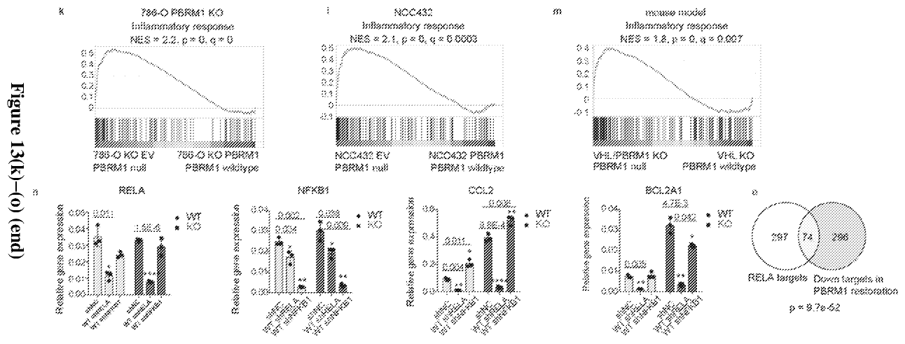

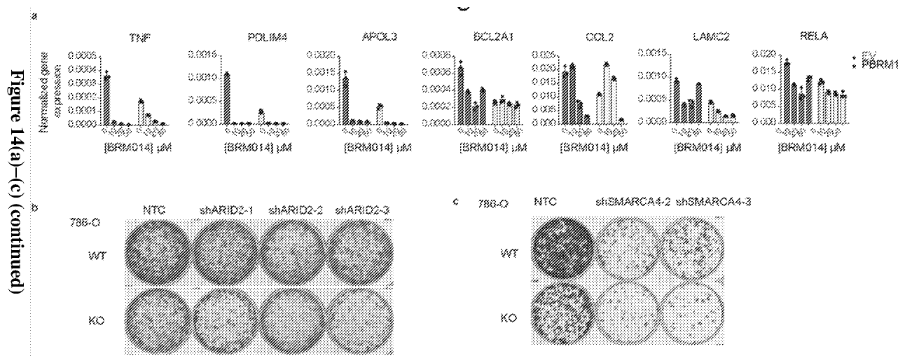

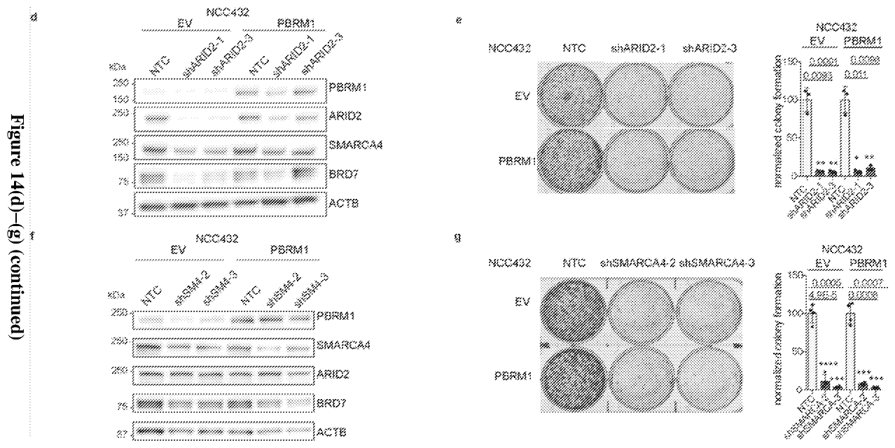

To gain biological insight into the tumor suppressive function of PB1, pathways most impacted by altered PBAF binding were examined. Genomic occupancy of PB1 was integrated with gene expression changes using BETA and it was observed that PB1 could act both as a transcriptional activator and a repressor (FIG. 13A). Because PB1 loss could impact regions other than its directly bound sites, the analysis was restricted to genes that could be rescued by re-expression of PB1 in 786-O PB1 KO cells (defined as p-value<0.05, |log fold change|>0.3). In total, 153 downregulated genes and 243 upregulated genes could be rescued by PB1 re-expression. Since there are 60% more upregulated genes compared to downregulated genes (FIG. 5A), PB1 loss may more frequently result in liberation of gene expression.

The most significant upregulated pathway after PB1 loss was HALLMARK_TNFA_SIGNALING_VIA_NFKB (q-value=0, hypergeometric test) (FIG. 5B). PB1 loss increased expression of NF-κB target genes in 786-O cells; conversely, PB1 restoration repressed gene expression in 786-O PB1 KO cells (NES=2.6, q<0.00002, GSEA) (FIG. 5C, 13B, C). Consistent changes in 3 additional isogenic tumor cell lines were seen: PB1 KO in PB1-proficient 769-P cells resulted in the upregulation of NF-κB target genes (NES=2.2, q<0.00001, GSEA) (FIG. 13D). Conversely, PB1 restoration repressed NF-κB pathway in NCC432 cell line (NES=1.7, q=0.017, GSEA) and A704 (NES=1.5, q=0.06, GSEA) (FIGS. 5D, 13E and 13F). Importantly, activation of NF-κB target genes was also observed in the genetic mouse model of VHL and PB1 double knockouts (NES=2.7, q<0.00002, GSEA) (FIG. 5E, 13G). Lastly, patient tumor samples from the TCGA collection harboring PB1 loss also significantly upregulated the pathway HALLMARK_TNFA_SIGNALING_VIA_NFKB compared to PB1-proficient tumors (NES=2.2, q<0.0001, GSEA) (FIG. 13H, I). Other pathways enriched in PB1-deficient tumors included HALLMARK_INTERFERON_GAMMA_RESPONSE and HALLMARK_HYPOXIA, consistent with the known effects of PB1 loss. PB1 loss led to profound target activation upon TNF stimulation in both 786-0 and NCC432 cells (FIG. 5F, FIG. 13J). Therefore, PB1 loss resulted in activation of NF-κB pathway at both the baseline and upon TNF stimulation across four isogenic cell lines, genetic mouse model and primary tumors.

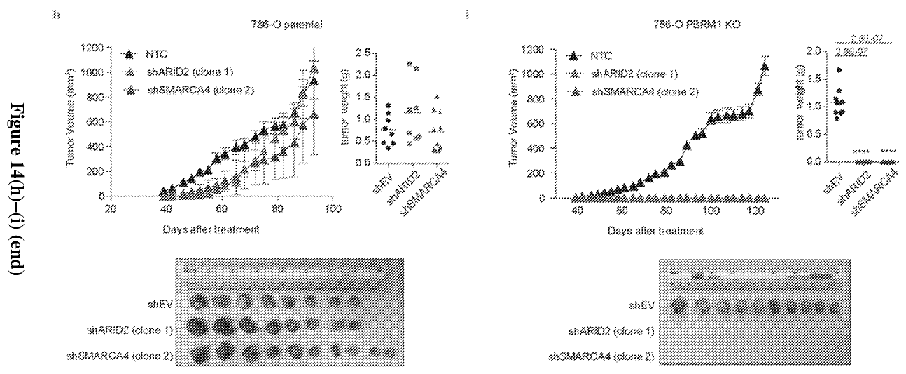

NF-κB coordinates with other transcription factors including STAT3 and IRF to regulate inflammation. Upregulation of immune-related pathways was observed, including inflammatory response, interferon gamma response, and IL6 via STAT3 (FIG. 5B, FIG. 13K-M). Target genes including ILIR2, CSF1, and IFNGR2 acquired increased binding of SMARCA4 and RELA. RELA gene encodes p65, a subunit of the canonical NF-κB complex. Gene expression of NF-κB targets was significantly reduced by RELA knockdown (FIG. 5G, 14B). RELA ChIP-seq was then performed to understand the effects of PB1 loss on RELA recruitment. Direct RELA targets were defined by overlapping RELA genomic occupancy and downregulated gene expression driven by shRELA (p<0.05 and log FC<−0.5). A third of the RELA target genes defined by these two criteria (113/371 genes) overlapped with upregulated genes after PB1 loss in 786-O cells (log 2FC>0.5 and p-value<0.05, negative binomial test) (FIG. 5H). PB1 restoration led to suppression of one fifth of the RELA target genes (log 2FC<−0.3 and p-value<0.5, negative binomial test, FIG. 14C). Therefore, a significant portion of gained RELA target genes was attributed to PB1 loss.

### NF-κB Activation Requires SMARCA4 and ARID2

It was previously shown that inhibiting the SMARCA4 ATPAse subunit abolished chromatin accessibility and binding of SMARCA4-dependent transcription factors. To understand whether the activation of pathway requires the ATPase activity of SMARCA4, 786-O and NCC432 cells were treated with SMARCA2/4 ATPase inhibitor. We observed dose-dependent downregulation of NF-κB target genes upon SMARCA42/4 ATPase inhibitor treatment. Some genes such as PDLIM4 and APOL3 were exquisitely sensitive to SMARCA2/4 ATPase inhibitor in both the PB1-proficient and deficient setting (FIG. 6A). Other genes such as BCL2A1 and TNF showed greater downregulation in the PB1-deficient setting compared to the proficient setting, consistently across 786-O and NCC432 (FIG. 13N). In 786-O parental and PB1 KO cells were treated with 10 μM of SMARCA2/4 ATPase inhibitor for 24 hours, gene set enrichment of the direct NF-κB targets shows greater effect of NF-κB downregulation in PB1-deficient cells compared to parental 786-O cells (FIG. 6B). This implies that the activation of the NF-κB pathway requires the ATPase subunit of SMARCA4.

To address the role of ARID2 in the activation of NF-κB pathway, ARID2 was transiently depleted using dox-inducible shRNA. Decreased levels of NF-κB targets (TNF, PDLIM4 and BCL2A1) were observed only in the PB1-deficient 786-O cells expressing ARID2 shRNA (FIG. 6D). As a second model, ARID2 was depleted in 769-P cells using CRISPR-mediated knockout. ARID2 depletion led to concomitant loss of PB1 and BRD7 in 769-P cells, again supporting the importance of ARID2 in maintaining protein stability of the entire PBAF complex (FIG. 6E). 769-P cells with ARID2 sgRNA (which is also PB1-deficient) still showed activation of NF-κB pathway compared to non-targeting control (NTC), although to a less degree than cells with sgPBRM1 (intact ARID2, NES=1.4, q=0.05, FIG. 6F). This implies that both ARID2 and SMARCA4 are important for the upregulation of NF-κB target genes.

SMARCA4 protein preferentially partnered with ARID2 but became disengaged from BRD7, which localized to other aberrant sites. ARID2 depletion by three independent shRNAs reduced colony formation in 786-O PB1-deficient cells (FIG. 12C). The requirement for ARID2 was similarly previously observed in tumorsphere assays. Depletion of ARID2 destabilized other PBAF subunits and led to concomitant decrease in the protein levels of PB1 and BRD7 (FIG. 6G, H). Similarly, SMARCA4 depletion also reduced colony formation in 786-O cells (FIG. 6I, J). It is possible that selected SMARCA4-deficient clones are able to eventually compensate for the loss of fitness with paralog protein SMARCA2, as in the case of 786-O SMARCA4 KO clones gaining growth advantage in vivo. Nonetheless, consistent decreased cell fitness in 786-O and NCC432 cells was observed at early passages (<2 passages) after SMARCA4 and ARID2 depletion by shRNA (FIG. 12E-H).

### PB1-Deficient PBAF Complexes Recruit RELA

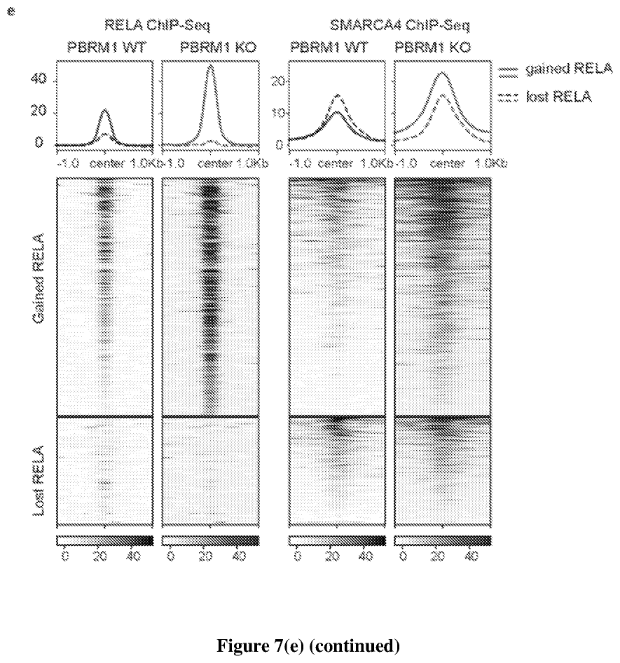

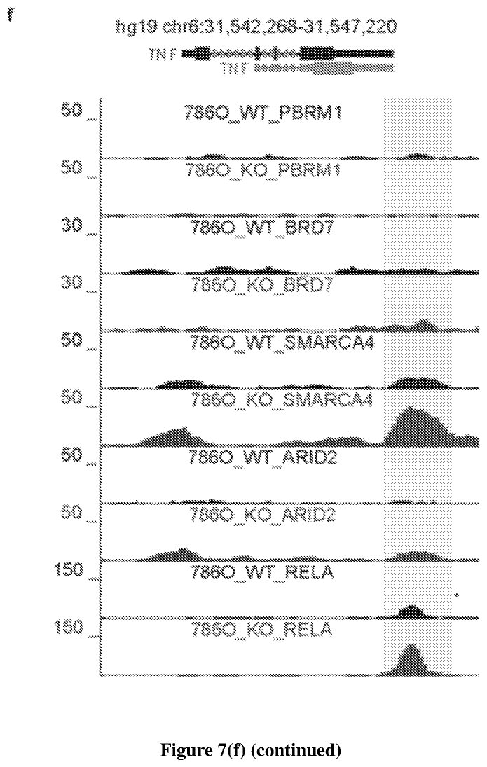

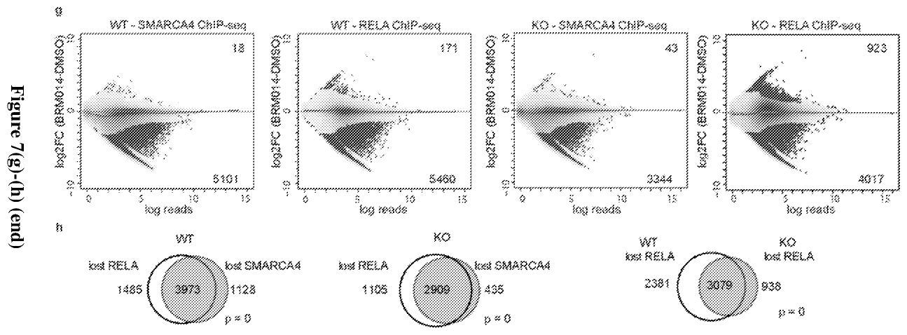

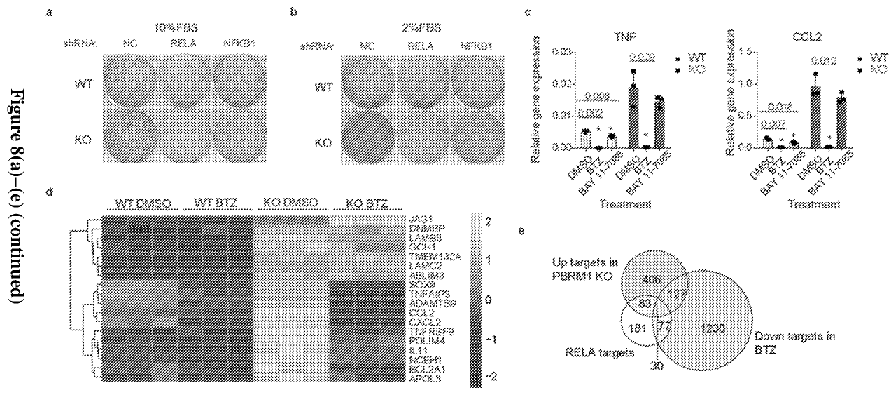

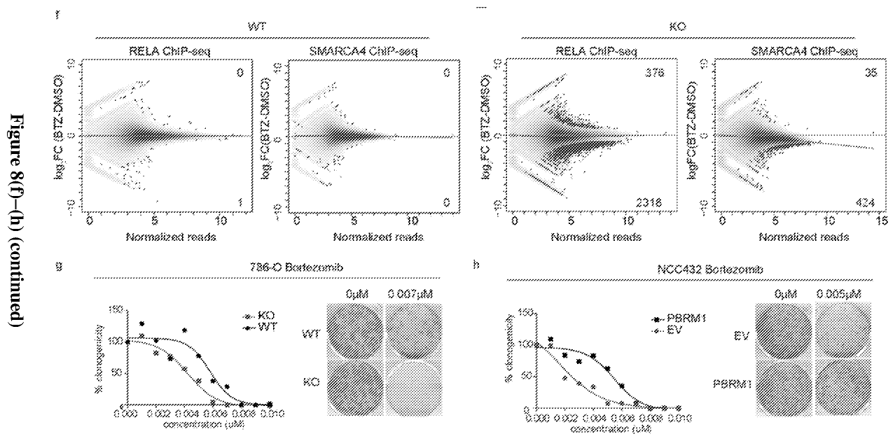

To draw a connection between NF-κB activation and altered PBAF binding, motif search in the newly acquired PBAF regions was performed. NF-κB p65 was the second most significantly enriched motif (p=1e-41) after TEAD (FIG. 7A). When the gained SMARCA4 peaks were mapped to their nearest TSS, NF-κB remained the top enriched pathway (p=1.7e-10, q=8.6e-09, hypergeometric test) followed by hypoxia, a previously known effect of PB1 loss (FIG. 7B). The same was observed with gained ARID2 gained peaks (p=0.007, q=0.36, hypergeometric test) and BRD7 gained peaks (BRD7 (p=4.4e-09, q=2.2e-07) (FIG. 14A), suggesting that PBAF subunits redistribute to regions that regulate the NF-κB pathway.

Mechanistically, PB1 loss did not significantly alter the nuclear localization, total protein levels or phosphorylation levels of NF-κB subunits (FIG. 7C). Instead, RELA acquired increased genomic occupancy at 689 regions, more than double the number of lost regions (306 regions) (FIG. 7C). Importantly, one-third of the gained RELA regions coincided with concomitant increased SMARCA4 occupancy (FIG. 7D, p=3.2e-61, hypergeometric test). RELA peaks also overlapped significantly with total ARID2 regions (p=0) or gained ARID2 regions (p=6.5e-18) (FIG. 14E). At the putative enhancers of many NF-κB target genes, significant co-enrichment of SMARCA4, RELA and ARID2 1 binding was seen (FIG. 7E, 7F, FIG. 14F). To conclusively determine whether SMARCA4 is necessary for RELA recruitment, SMARCA4 was inactivated by ATPase inhibitor and its effect on RELA binding was examined. Strikingly, it was observed that ATPase inhibitor treatment led to significantly decreased RELA occupancy at 5/6 regions probed in the PB1-deficient setting (FIG. 7G). Therefore, the ATPase function of SMARCA4 in the residual PBAF complexes enhances the recruitment of nuclear factor NF-κB to the chromatin.

### NF-κB Inhibitor Bortezomib Preferentially Targets PB1-Deficient Tumors

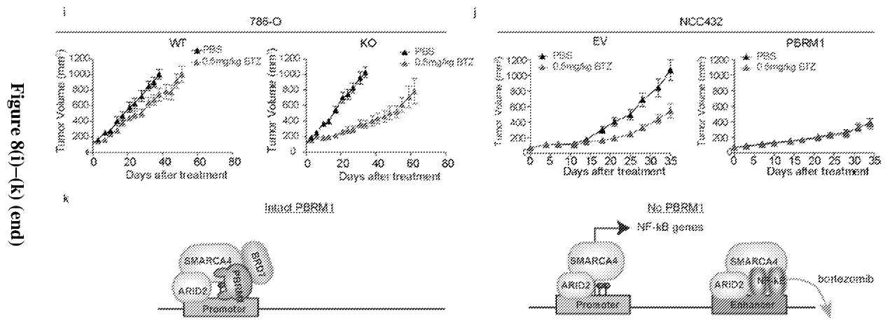

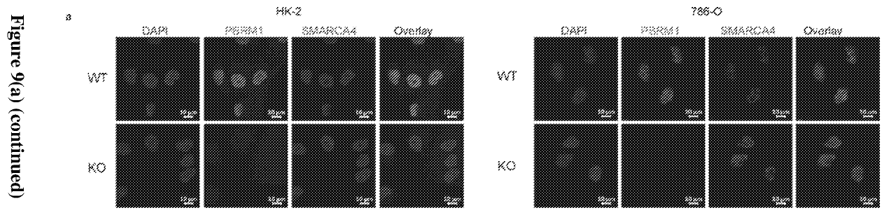

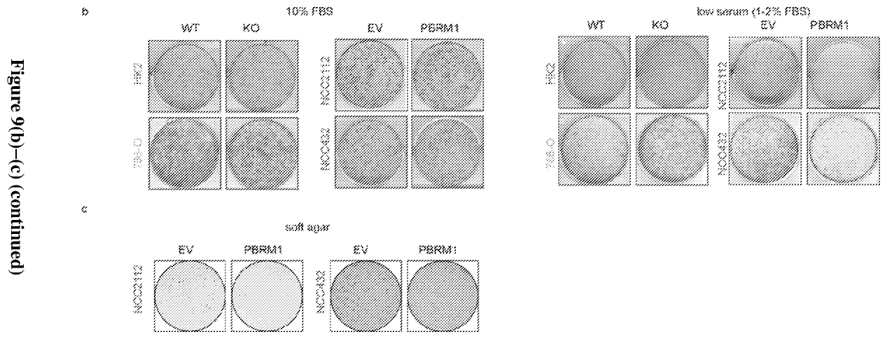

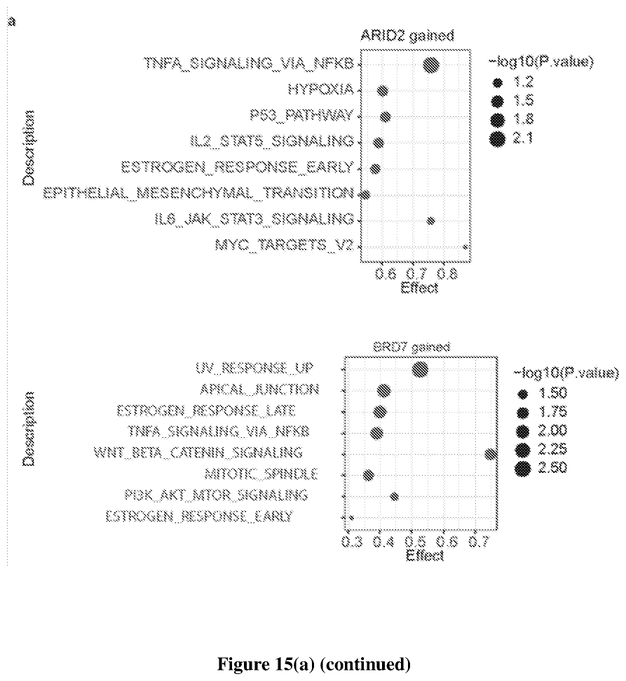

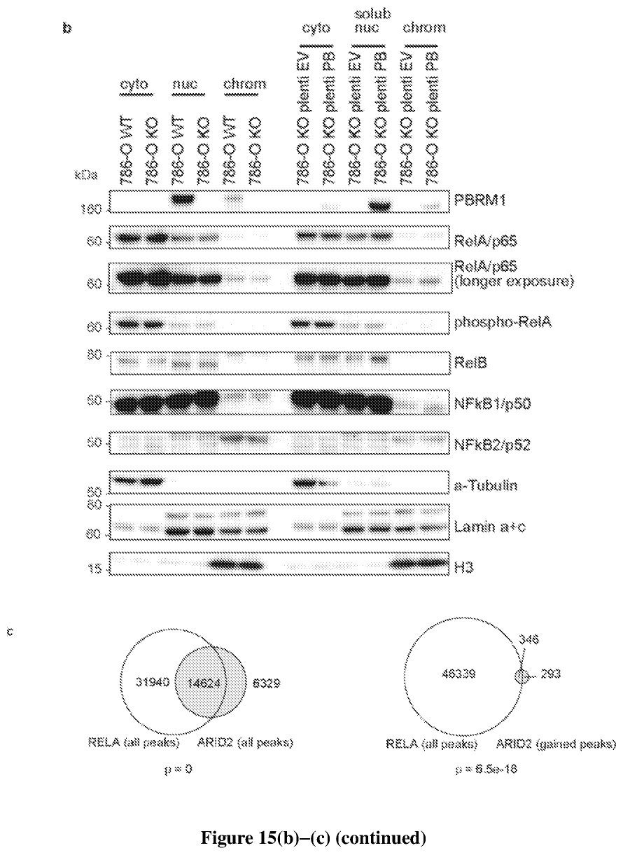

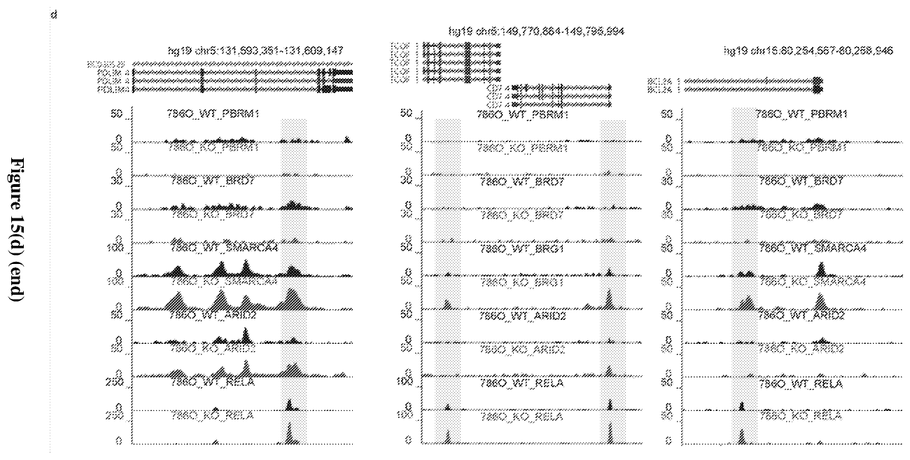

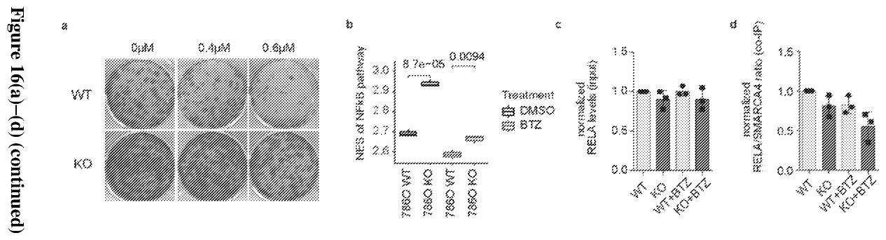

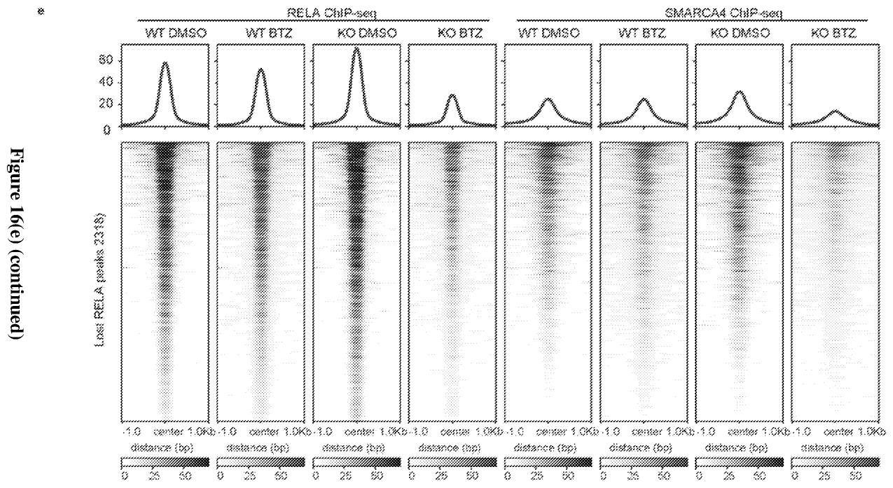

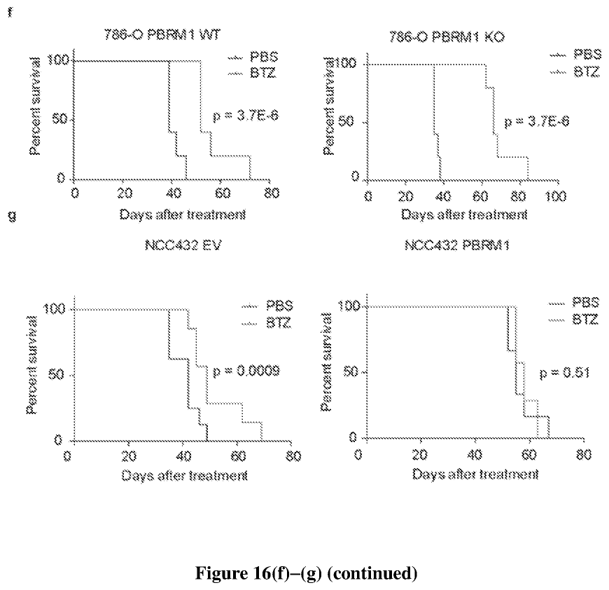

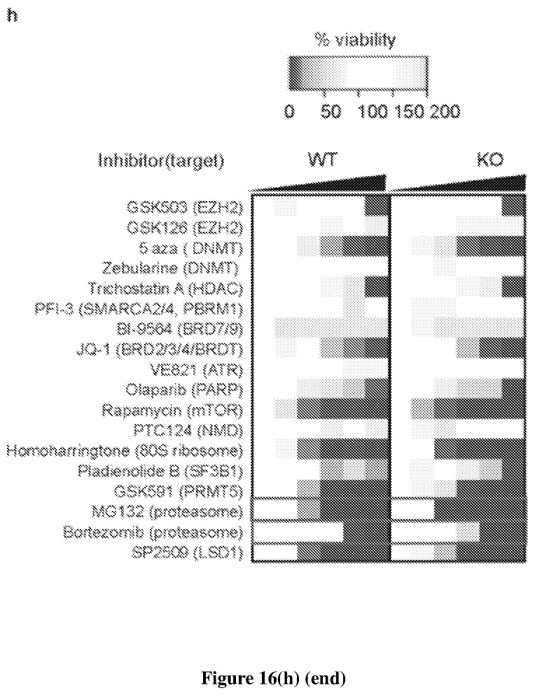

Since NF-κB activation was the most significantly upregulated pathway after PB1 loss, the effects of inhibiting NF-κB on tumor formation were examined. Knockdown of RELA (less so NFKB1) reduced colony formation under both 10% and 2% serum conditions (FIG. 8A, B). Two NF-κB inhibitors, bortezomib and Bay 11-7085, were investigated. The proteasome inhibitor bortezomib induces nuclear translocation and accumulation of IκBα which then associates with nuclear NF-κB and inhibits its DNA binding activity. Bay 11-7085 inhibits TNFα-induced but not basal IκBα phosphorylation and prevents TNFα-induced nuclear translocation of NF-κB. Only bortezomib but not Bay 11-7085 was effective at suppressing the expression of NF-κB target genes and reducing colony formation (FIG. 8C, FIG. 15A). Analysis of global transcriptomics by RNA-seq validated the downregulation of the NF-κB pathway by bortezomib treatment (FIG. 8D, FIG. 15B). In addition to RELA targets, bortezomib also suppressed additional 127 targets upregulated by PB1 loss, enriched in inflammatory response (ZHOU_INFLAMMATORY_RESPONSE_FIMA_UP, q=8.8e-4) and oncogenic pathway (DODD_NASOPHARYNGEAL_CARCINOMA_UP, q=4.0e-3) (FIG. 8E).

Bortezomib treatment did not significantly affect the interaction between SMARCA4 and RELA or the total protein levels of RELA (FIG. 8F, FIG. 15C). Instead, ChIP-qPCR of RELA showed profound reduction of RELA occupancy on the chromatin after bortezomib treatment in the PB1 KO 786-O cells, and to a less extent in the PB1 WT cells.

Bortezomib resulted in preferential reduction of colony formation in PB1-deficient 786-O and NCC432 compared to PB1-proficient isogenic cells (FIG. 8G, H). This reduction in colony formation was recapitulated in vivo-mice bearing PB1-deficient 786-O or NCC432 responded better to bortezomib compared to PB1-proficient isogenic cells (FIG. 8I, J), and survived for a longer duration compared to those mice bearing PB1-proficient cells (FIG. 15D, 15E). While PB1 deficiency failed to sensitize tumor cells to common epigenetic drugs including EZH2i, DNMTi, HDACi, BETi and PRMT5i (FIG. 15F), inhibition of NF-κB using proteasome inhibitors is a potential therapeutic strategy against PB1-deficient tumors.

### Example 2

PBRM1 is mutated in ~40% of all clear cell renal cell carcinomas but the epigenomic consequences of its deficiency have not been well studied. It is now demonstrated that in VHL deficient ccRCC, PB1 deficiency alters the molecular composition of the PBAF complex by disengaging BRD7 from SMARCA4 but preserving the association between ARID2 and SMARCA4 on the chromatin. The loss of PB1-BRD7 submodule compromises the suppressive function of the PBAF complex by disrupting the targeting specificity of the PBAF complexes leading to aberrant binding of residual PBAF complexes at de novo regions and enhanced recruitment of NF-κB complexes by SMARCA4 (FIG. 8K). Proteasome inhibitor bortezomib reverses NF-κB activation and selectively targets PB1-deficient tumors.

The genomic consequences of PB1 loss differ vastly from other subunits of the SWI/SNF complex and points towards indirect gain of function. The loss of SMARCB1 in rhabdoid tumors destabilizes the entire BAF complexes and evicts SMARCA4 and SMARCC1 from typical enhancers but retains them at super-enhancer of developmental genes. The loss of ARID1A also leads to reduced SMARCA4 and SMARCC1 binding at lineage-specifying genes in breast cancer. In contrast, PB1-deficient complexes remain largely intact, with SMARCA4 and ARID2 substituting for the lack of PB1 at previously bound PB1 sites. The most profound changes after PB1 loss occur at de novo regions away from PB1 binding sites, resulting in transcriptional activation of pro-oncogenic NF-κB target genes driven by this PB1/BRD7-deficient PBAF complex. Therefore, PB1 serves as the anchorage to lock BRD7 and the rest of PBAF complex at gene promoters and prevent aberrant recruitment of transcription factors.

NF-κB has been identified as a major pathway activated upon PB1 loss in human cancer cell line models and genetic mouse model. Importantly, NF-κB activation by PB1 loss differs from other modes of NF-κB activation (increased nuclear translocation and NF-kB phosphorylation) and indeed relies on increased chromatin recruitment by SMARCA4 and depends on its ATPase function. SMARCA4 was known to form complexes with NF-κB to regulate Schwann cell differentiation. NF-κB promotes all aspects of cancer hallmarks, including enhancing tumor proliferation, epithelial-mesenchymal transition, angiogenesis and circumventing apoptosis. Cancer-cell-intrinsic inflammation mediated by NF-κB drives ccRCC metastasis. Furthermore, since NF-κB is a critical mediator of inflammatory response, NF-κB activation by PB1 loss may play a direct role in activating other immune-related pathways such as interferon gamma response and IL-6 JAK-STAT3 signaling. It remains to be investigated whether NF-κB upregulation is responsible for the improved response to checkpoint blockade in patients with PBRM1-deficient tumors, and whether a specific NF-κB signature may further stratify patient response.

NF-κB activation through TNF is a targetable vulnerability in PBRM1-deficient tumors. The use of bortezomib to suppress NF-κB activity and inhibit tumor growth was explored. A comprehensive literature survey of 32 trials using bortezomib as a monotherapy ranked RCC as the most responsive tumor type amongst all solid tumors. In a phase II trial of 37 patients with metastatic RCC, 4 patients achieved partial response to bortezomib monotherapy and 14 patients achieved stable disease. Another independent study showed RCC patients having the best response to bortezomib plus bevacizumab across 14 solid tumor types. The best responder to this combined therapy failed prior bevacizumab therapy but saw tumor shrinkage of 88% with bevacizumab and bortezomib combined therapy, implying that bortezomib was the crucial factor responsible for this tumor regression. Combination treatment with bortezomib may be explored in patients harboring PB1-deficient tumors and enhanced NF-κB activity. The primary function of PB1 is to safeguard chromatin by preventing the emergence of aberrantly targeted PBAF complexes and activation of pro-tumorigenic NF-κB target genes. Together with the VHL tumor suppressor that maintains enhancer integrity, PB1 serves as the guardian of the epigenome in renal epithelial cells.

It will be appreciated that many further modifications and permutations of various aspects of the described embodiments are possible. Accordingly, the described aspects are intended to embrace all such alterations, modifications, and variations that fall within the spirit and scope of the appended claims.

1-16. (canceled)

17. A method of treating a cancer in a subject, the method comprising a) detecting an indicator that is indicative of a level or activity of PB1 in a sample obtained from the subject, wherein the cancer is identified as being likely to be responsive to proteasome inhibitor when the level or activity of PB1 indicated by the indicator corresponds to an absence or decreased level or activity of PB1 in the sample as compared to a control; and b) administering the proteasome inhibitor to the subject when the subject is found likely to be responsive to treatment with the proteasome inhibitor.

18. The method of claim 17, wherein the method comprises comparing the indicator to the control.

19. The method of claim 17, wherein the indicator is a PB1 transcript level, PB1 protein level, or a variation in the PB1 gene sequence or operably connected transcriptional control region or UTR.

20. The method of claim 17, wherein Von Hippel-Lindau (VHL) is absent in the cancer or wherein the cancer has a decreased expression or activity of Von Hippel-Lindau (VHL).

21. The method of claim 17, wherein the cancer is a renal cancer.

22. The method of claim 21, wherein the renal cancer is renal cell carcinoma.

23. The method of claim 17, wherein the sample is a cancer or blood sample.

24. The method of claim 17, wherein the proteasome inhibitor is bortezomib or carfilzomib.

25. A method of treating a cancer in a subject, the method comprising a) selecting the subject found likely to be responsive to treatment with a proteasome inhibitor based on an indicator that is indicative of a level or activity of PB1 that has been detected in a sample obtained from the subject, wherein the cancer is identified as being likely to be responsive to proteasome inhibitor when the level or activity of PB1 indicated by the indicator corresponds to an absence or decreased level or activity of PB1 in the sample as compared to a control; and b) administering the proteasome inhibitor to the subject when the subject is found likely to be responsive to treatment with the proteasome inhibitor.

26. A method of inhibiting proliferation of a cancer cell, the method comprising contacting the cancer cell with a proteasome inhibitor, wherein PB1 is absent in the cancer cell or wherein the cancer cell has a decreased level or activity of PB1 as compared to a control.

27. The method of claim 26, wherein Von Hippel-Lindau (VHL) is absent in the cancer cell or wherein the cancer cell has a decreased expression or activity of Von Hippel-Lindau (VHL).

28. The method of claim 26, wherein the cancer is a renal cancer.

29. The method of claim 27, wherein the renal cancer is renal cell carcinoma.

30. The method of claim 26, wherein the proteasome inhibitor is bortezomib or carfilzomib.


SEQUENCE LISTING (23 sequences)

SEQ ID NO: 1 - protein, 1689 aa, Homo sapiens
MGSKRRRATSPSSSVSGDFDDGHHSVSTPGPSRKRRRLSNLPTVDPIAVCHELYNTIRDYKDEQGRLLCELFIRAPKRRNQPDYYEVVSQPIDLMKIQQKLKMEEYDDVNLLTADFQLLFNNAKSYYKPDSPEYKAACKLWDLYLRTRNEFVQKGEADDEDDDEDGQDNQGTVTEGSSPAYLKEILEQLLEAIVVATNPSGRLISELFQKLPSKVQYPDYYAIIKEPIDLKTIAQRIQNGSYKSIHAMAKDIDLLAKNAKTYNEPGSQVFKDANSIKKIFYMKKAEIEHHEMAKSSLRMRTPSNLAAARLTGPSHSKGSLGEERNPTSKYYRNKRAVQGGRLSAITMALQYGSESEEDAALAAARYEEGESEAESITSFMDVSNPFYQLYDTVRSCRNNQGQLIAEPFYHLPSKKKYPDYYQQIKMPISLQQIRTKLKNQEYETLDHLECDLNLMFENAKRYNVPNSAIYKRVLKLQQVMQAKKKELARRDDIEDGDSMISSATSDTGSAKRKSKKNIRKQRMKILFNVVLEAREPGSGRRLCDLFMVKPSKKDYPDYYKIILEPMDLKIIEHNIRNDKYAGEEGMIEDMKLMFRNARHYNEEGSQVYNDAHILEKLLKEKRKELGPLPDDDDMASPKLKLSRKSGISPKKSKYMTPMQQKLNEVYEAVKNYTDKRGRRLSAIFLRLPSRSELPDYYLTIKKPMDMEKIRSHMMANKYQDIDSMVEDFVMMFNNACTYNEPESLIYKDALVLHKVLLETRRDLEGDEDSHVPNVTLLIQELIHNLFVSVMSHQDDEGRCYSDSLAEIPAVDPNFPNKPPLTFDIIRKNVENNRYRRLDLFQEHMFEVLERARRMNRTDSEIYEDAVELQQFFIKIRDELCKNGEILLSPALSYTTKHLHNDVEKERKEKLPKEIEEDKLKREEEKREAEKSEDSSGAAGLSGLHRTYSQDCSFKNSMYHVGDYVYVEPAEANLQPHIVCIERLWEDSAGEKWLYGCWFYRPNETFHLATRKFLEKEVFKSDYYNKVPVSKILGKCVVMFVKEYFKLCPENFRDEDVFVCESRYSAKTKSFKKIKLWTMPISSVRFVPRDVPLPVVRVASVFANADKGDDEKNTDNSEDSRAEDNFNLEKEKEDVPVEMSNGEPGCHYFEQLHYNDMWLKVGDCVFIKSHGLVRPRVGRIEKVWVRDGAAYFYGPIFIHPEETEHEPTKMFYKKEVFLSNLEETCPMTCILGKCAVLSFKDFLSCRPTEIPENDILLCESRYNESDKQMKKFKGLKRFSLSAKVVDDEIYYFRKPIVPQKEPSPLLEKKIQLLEAKFAELEGGDDDIEEMGEEDSEVIEPPSLPQLQTPLASELDLMPYTPPQSTPKSAKGSAKKEGSKRKINMSGYILFSSEMRAVIKAQHPDYSFGELSRLVGTEWRNLETAKKAEYEERAAKVAEQQERERAAQQQQPSASPRAGTPVGALMGVVPPPTPMGMLNQQLTPVAGMMGGYPPGLPPLQGPVDGLVSMGSMQPLHPGGPPPHHLPPGVPGLPGIPPPGVMNQGVAPMVGTPAPGGSPYGQQVGVLGPPGQQAPPPYPGPHPAGPPVIQQPTTPMFVAPPPKTQRLLHSEAYLKYIEGLSAESNSISKWDQTLAARRRDVHLSKEQESRLPSHWLKSKGAHTTMADALWRLRDLMLRDTLNIRQAYNLENV

SEQ ID NO: 2 - protein, 213 aa, Homo sapiens
MPRRAENWDEAEVGAEEAGVEEYGPEEDGGEESGAEESGPEESGPEELGAEEEMEAGRPRPVLRSVNSREPSQVIFCNRSPRVVLPVWLNFDGEPQPYPTLPPGTGRRIHSYRGHLWLFRDAGTHDGLLVNQTELFVPSLNVDGQPIFANITLPVYTLKERCLQVVRSLVKPENYRRLDIVRSLYEDLEDHPNVQKDLERLTQERIAHQRMGD

SEQ ID NO: 3 - DNA, 23 bp, synthetic construct
  source 1..23: mol_type = other DNA
caccgcgaggagatctatatctt

SEQ ID NO: 4 - DNA, 25 bp, synthetic construct
  source 1..25: mol_type = other DNA
caccgaccttccatttcgctcagtt

SEQ ID NO: 5 - DNA, 22 bp, synthetic construct
  source 1..22: mol_type = other DNA
tggctgcagccaagttggatgg

SEQ ID NO: 6 - DNA, 22 bp, synthetic construct
  source 1..22: mol_type = other DNA
ataacacctaacctaacatggg

SEQ ID NO: 7 - DNA, 22 bp, synthetic construct
  source 1..22: mol_type = other DNA
ataggcatttgatagtacttgg

SEQ ID NO: 8 - DNA, 22 bp, synthetic construct
  source 1..22: mol_type = other DNA
gttaccaatacaattgcacagg

SEQ ID NO: 9 - DNA, 22 bp, synthetic construct
  source 1..22: mol_type = other DNA
attgacatgggtttagcgttgg

SEQ ID NO: 10 - DNA, 22 bp, synthetic construct
  source 1..22: mol_type = other DNA
aagggatcctaggatatcctgg

SEQ ID NO: 11 - DNA, 22 bp, synthetic construct
  source 1..22: mol_type = other DNA
tggctgcagccaagttggatgg

SEQ ID NO: 12 - DNA, 22 bp, synthetic construct
  source 1..22: mol_type = other DNA
cctctctaagatgtaaactagg

SEQ ID NO: 13 - DNA, 22 bp, synthetic construct
  source 1..22: mol_type = other DNA
atctatcaagctacctgctagg

SEQ ID NO: 14 - DNA, 22 bp, synthetic construct
  source 1..22: mol_type = other DNA
agggtccaatcaggctatgagg

SEQ ID NO: 15 - DNA, 22 bp, synthetic construct
  source 1..22: mol_type = other DNA
atctatcaagctacctgctagg

SEQ ID NO: 16 - DNA, 22 bp, synthetic construct
  source 1..22: mol_type = other DNA
agggtccaatcaggctatgagg

SEQ ID NO: 17 - DNA, 22 bp, synthetic construct
  source 1..22: mol_type = other DNA
ctgtataaaccgagggcgacgg

SEQ ID NO: 18 - DNA, 22 bp, synthetic construct
  source 1..22: mol_type = other DNA
agggtccaatcaggctatgagg

SEQ ID NO: 19 - DNA, 57 bp, synthetic construct
  source 1..57: mol_type = other DNA
ccggccagcgtgaaatgtatccattctcgagaatggatacatttcacgctggttttt

SEQ ID NO: 20 - DNA, 57 bp, synthetic construct
  source 1..57: mol_type = other DNA
ccggcgtacctgtcttcgtttcctactcgagtaggaaacgaagacaggtacgttttt

SEQ ID NO: 21 - DNA, 57 bp, synthetic construct
  source 1..57: mol_type = other DNA
ccggccgactaacagctgccttaatctcgagattaaggcagctgttagtcggttttt

SEQ ID NO: 22 - DNA, 57 bp, synthetic construct
  source 1..57: mol_type = other DNA
ccggtcaaacagtaccagatcaaagctcgagctttgatctggtactgtttgattttt

SEQ ID NO: 23 - DNA, 57 bp, synthetic construct
  source 1..57: mol_type = other DNA
ccggctttgcgtatcgcggctttaactcgagttaaagccgcgatacgcaaagttttt